(19) United States
(12) Patent Application Publication
(10) Pub. No.: US 2025/0146081 A1
(43) Pub. Date: May 8, 2025

(54) TCR/BCR PROFILING FOR CELL-FREE NUCLEIC ACID DETECTION OF CANCER

(71) Applicant: Freenome Holdings, Inc., South San Francisco, CA (US)

(72) Inventors: Richard BOURGON, San Francisco, CA (US); Brian O’DONOVAN, Spokane, WA (US); Anupriya TRIPATHI, San Francisco, CA (US); Francesco VALLANIA, San Francisco, CA (US); David WEINBERG, Mill Valley, CA (US)

(21) Appl. No.: 18/892,913

(22) Filed: Sep. 23, 2024

Related U.S. Application Data
(63) Continuation of application No. PCT/US2023/016044, filed on Mar. 23, 2023.
(60) Provisional application No. 63/323,577, filed on Mar. 25, 2022.

(51) Int. Cl. C12Q 1/6886; C12Q 1/6869; G16B 40/00

(52) U.S. Cl. CPC C12Q 1/6886; C12Q 1/6869; G16B 40/00; C12Q 2600/118; C12Q 2600/158

(57) ABSTRACT

Methods and systems are provided to obtain a clinically meaningful characterization of T cell receptor (TCR) or B cell receptor (BCR) repertoire using cell-free-DNA or immune cell-derived DNA.

### CROSS-REFERENCE

This application is a continuation of International Application No. PCT/US2023/016044, filed Mar. 23, 2023, which claims the benefit of U.S. Provisional Application No. 63/323,577, filed Mar. 25, 2022, each of which is incorporated by reference herein in its entirety.

### BACKGROUND

There is uncertainty in use of cfDNA samples for immune repertoire analysis because signals and library complexity in cfDNA samples can be easily lost during processing operations prior to sequencing. This is particularly problematic for early-stage cancer detection where low tumor burden samples may not have sufficiently detectable levels of tumor indicative cfDNA required for multiomic analysis.

### SUMMARY

There is a need for improved methods to utilize plasma cfDNA sequencing data by leveraging TCR and BCR sequences in the sample that may alone, or in combination with other liquid biopsy assays, provide meaningful applications for early cancer detection. Accordingly, methods and systems are provided herein to obtain a clinically meaningful characterization of T cell receptor (TCR) and/or B cell receptor (BCR) repertoire using cell-free-DNA and/or immune cell-derived DNA. Profiling of TCR and BCR expression associated with cell proliferative disorder is used for methods of cancer detection and tracking disease progression. TCR and BCR sequences capture underexplored biomarkers for early detection of cancer and/or patient prognosis regarding their cancer progression. In some cases, analysis of peripheral T cell receptor (TCR) and B cell receptor (BCR) repertoire using a deep learning algorithm may provide a basis for detection of asymptomatic patients with early-stage cancer. Deep learning machine learning applications integrate datasets from peripheral TCR and BCR repertoire sequencing for classification of biological samples from subject populations. Using probes to capture cfDNA fragments from TCR complementarity-determining region 3 (CDR3), as required for the deep learning machine learning algorithms.

Also provided are methods and systems directed to computational features capturing different aspects of T-cell and B-cell repertoires present in cfDNA and use thereof in methods of disease detection such as cancer detection.

Characterization and profiling of T-cell receptor expression may be beneficial for the classification of individuals with cancer alone or in combination with a multiomic analysis approach to cell-free nucleic acid analysis.

In an aspect, the present disclosure provides a method for sequencing a biological sample from an individual comprising:
a) obtaining a nucleic acid from the biological sample;
b) contacting the nucleic acid with complementary oligonucleotides to regions upstream and downstream to the CDR3 domain wherein the complementary oligonucleotides are sequencing substantially across the CDR3 regions in the sample; and
c) generating CDR3 nucleic acid sequence data from the nucleic acid.

In one embodiment, the biological sample is selected from a sample of cell-free nucleic acid, plasma, serum, whole blood, buffy coat, single cell or tissue.

In one embodiment, the complementary oligonucleotides are modified to permit sequencing after enzymatic conversion for methylation sequencing.

In one embodiment, the complementary oligonucleotides are designed separately against both C-to-T/G-to-A converted strands of DNA and accounting for CpG's being completely methylated or unmethylated.

In one embodiment, the complementary oligonucleotides are selected to be complementary to regions proximal to the V-D junction and/or fully overlap the J region.

In one embodiment, the generating CDR3 nucleic acid sequence data is performed on targeted nucleic acid regions or whole genome sequencing methods

In one embodiment, the method further comprises sequencing a CDR3 domain from PBMCs from the same individual obtained at the same time as the sample of cell-free nucleic acid.

In one embodiment, the method further comprises applying a computational analysis on the CDR3 nucleic acid sequence data to produce a T cell receptor (TCR) and/or B cell receptor (BCR) profile of the individual.

In one embodiment, the computational analysis further comprises removing non-CDR3 sequence information from the CDR3 nucleic acid sequence data.

In one embodiment, the computational analysis further comprises a PCA, CNN, MiXCR, TRUST, V′DJer, or DeepCAT method.

In various embodiments, TCR and BCR profiles are associated with the presence of lung, colon, liver, ovarian, pancreatic, prostate, rectal, and/or breast cell proliferative disorders or progression thereof.

In an aspect, the present disclosure provides a method for sequencing a sample of cell-free nucleic acid from an individual comprising:
a) obtaining a sample comprising a cell-free nucleic acid;
b) contacting the cell-free nucleic acid from the sample with complementary oligonucleotides to regions upstream and downstream to the CDR3 domain wherein the complementary oligonucleotides are sequencing substantially across the CDR3 regions in the sample; and
c) generating CDR3 nucleic acid sequence data from the cell-free nucleic acid sample.

In an aspect, the present disclosure provides a method for detecting cancer in an individual T cell receptor and/or B cell receptor expression profile in a biological sample from an individual comprising:
a) obtaining a cell-free nucleic acid from the biological sample;
b) contacting the nucleic acid from the biological sample with complementary oligonucleotides to regions upstream and downstream to the CDR3 domain, wherein the complementary oligonucleotides are sequencing substantially across the CDR3 regions in the sample to generate CDR3 nucleic acid sequence data;
c) applying a computational analysis to the CDR3 nucleic acid sequence data to produce the T cell receptor profile or B cell receptor profile in the sample; and
d) applying a machine learning model trained on T cell receptor and/or B cell receptor expression profiles to the T cell receptor and/or B cell receptor profile to classify individuals with or without cancer.

In one embodiment, the complementary oligonucleotides are modified to permit sequencing after enzymatic conversion for methylation sequencing.

In one embodiment, the complementary oligonucleotides are designed separately against both C-to-T/G-to-A converted strands of DNA and accounting for CpG's being completely methylated or unmethylated.

In one embodiment, the complementary oligonucleotides are selected to be complementary to regions proximal to the V-D junction and/or fully overlap the J region.

In one embodiment, the generating CDR3 nucleic acid sequence data is performed on targeted nucleic acid regions or whole genome sequencing methods

In one embodiment, the method further comprises sequencing a CDR3 domain from PBMCs from the same individual obtained at the same time as the sample of cell-free nucleic acid.

In one embodiment, the method further comprises analyzing one or more of genomic, methylomic, transcriptomic, proteomic or metabolomic information in the biological sample from the individual.

In one embodiment, the one or more of genomic, methylomic, transcriptomic, proteomic or metabolomic information in the biological sample from the individual is included in training the machine learning model trained on T cell receptor expression.

In one embodiment, the computational analysis further comprises removing non-CDR3 sequence information from the CDR3 nucleic acid sequence data.

In one embodiment, the computational analysis further comprises a PCA, CNN, MiXCR, TRUST, V′DJer, or DeepCAT method performed on the T cell and/or B cell receptor sequences.

In one embodiment, the trained machine learning model is a classifier trained to distinguish between individuals with or without cancer.

In an aspect, the present disclosure provides a method for identifying prognostic or predictive biomarkers in an individual T cell receptor and/or B cell receptor expression profile in a sample of cell-free nucleic acid from an individual comprising:
a) obtaining a sample comprising cell-free nucleic acids;
b) contacting a cell-free nucleic acid from the sample with complementary oligonucleotides to regions upstream and downstream to the CDR3 domain,
wherein the complementary oligonucleotides are sequencing substantially across the CDR3 regions in the sample to generate CDR3 nucleic acid sequence data; and
c) applying a computational analysis on the CDR3 nucleic acid sequence data to identify prognostic or predictive biomarkers in the sample.

In an aspect, the present disclosure provides a system for sequencing a sample of cell-free nucleic acid from an individual, the system comprising one or more processors and memory operatively coupled to the one or more processors, wherein the one or more processors are programmed to:
a) obtain a sample comprising a cell-free nucleic acid;
b) contact the nucleic acid with complementary oligonucleotides to regions upstream and downstream to the CDR3 domain wherein the complementary oligonucleotides are sequencing substantially across the CDR3 regions in the sample; and
c) generate CDR3 nucleic acid sequence data from the nucleic acid.

In one embodiment, the complementary oligonucleotides are modified to permit sequencing after enzymatic conversion for methylation sequencing.

In one embodiment, the complementary oligonucleotides are designed separately against both C-to-T/G-to-A converted strands of DNA and accounting for CpG's being completely methylated or unmethylated.

In one embodiment, the complementary oligonucleotides are selected to be complementary to regions proximal to the V-D junction and/or fully overlap the J region.

In one embodiment, the generating CDR3 nucleic acid sequence data is performed on targeted nucleic acid regions or whole genome sequencing methods.

In one embodiment, the one or more processors are programmed to further sequence a CDR3 domain from PBMCs from the same individual obtained at the same time as the sample of cell-free nucleic acid.

In one embodiment, the computational analysis further comprises removing non-CDR3 sequence information from the sequence data.

In one embodiment, the computational analysis further comprises PCA, CNN, DeepCAT methods.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF DRAWINGS

Examples of the present disclosure will now be described, by way of example only, with reference to the attached Figures. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 provides a schematic of a computer system that is programmed or otherwise configured with the machine learning models and classifiers in order to implement methods provided herein.

FIGS. 2A-2D provide a schematic showing VDJ region sequencing. V, D, J segments exist in germline genome, and sequencing primers across the variable CDR3 region are used to obtain substantially complete sequencing of the CDR3 region.

FIG. 3 provides schematics showing VDJ region sequencing, including library preparation (including a first PCR amplification and a second PCR amplification), sequencing, and performing CDR3 and VDJ assignments (e.g., using MiXCR).

FIG. 4 provides plots showing a number of unique CDR3s detected per input mass of buffy coat genomic DNA (gDNA).

FIG. 5 provides plots showing a number of unique CDRs vs. sampling depth (e.g., in-silico downsampling of productive sequences) for various input masses (e.g., 10 ng, 100 ng, 250 ng, 500 ng, 1000 ng, and 1500 ng).

FIGS. 6A-6C provide plots showing comparisons between technical replicates of each of the first and second PCR amplification operations (FIGS. 6A-6B) and a Venn diagram indicating overlaps between three replicates of PCR amplification (FIG. 6C).

FIG. 7 provides plots showing recovery of spiked-in Jurkat gDNA (percent Junkat) and detection of spiked-in Jurkat gDNA (percent Jurkat clones detected using MiXCR).

FIG. 8 provides a plot showing a Venn diagram indicating overlaps between gDNA samples from three healthy donor subjects.

FIGS. 9A-9C provide plots showing comparisons between number of unique CDR3s, CDR3 length distribution (productive), and CDR3 frequency distribution (productive) between a healthy donor gDNA vs. cell-free DNA (FIG. 9A); comparisons between number of unique CDR3s that are productive or not productive across four donor subjects (FIG. 9B); and comparisons of productive sequences in gDNA samples across four donor subjects (FIG. 9C).

FIGS. 10A-10B provide plots showing Jurkat spike-in recovery results (detected clone fraction vs. spike-in fraction) (FIG. 10A), and comparisons between Spearman correlation, Jaccard similarity, and modified Jaccard similarity metrics between a workflow of the present disclosure (Freenome) and an alternative sequencing workflow (Adaptive) (FIG. 10B).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure relates generally to cancer detection and disease monitoring. More particularly, the field relates to cancer-related T-cell receptor expression detection and disease monitoring in early-stage colorectal cancer. Cancer screening and monitoring may help to improve outcomes over the past few decades because early detection leads to a better outcome as the cancer may be eliminated before it has spread. In the case of colorectal cancer, for instance, the use of colonoscopy may play a role in improving early diagnosis. Unfortunately, there may be challenges that arise due to patient compliance with screening not being adequate at recommended regularity.

A primary issue for any screening tool may be the compromise between false positive and false negative results (or specificity and sensitivity) which lead to extraneous investigations in the former case, and ineffectiveness in the latter case. An ideal test may be one that has a high Positive Predictive Value (PPV), minimizing extraneous investigations but detecting the vast majority of cancers. Another key factor may be what is called “detection sensitivity”, to distinguish it from test sensitivity, and that is the lower limits of detection in terms of the size of the tumor. Unfortunately, waiting for a tumor to grow to a size large enough to release circulating tumor markers at levels sufficient for detection may contradict the requirement for early detection in order to treat a tumor as stages where treatments are most effective. Hence, there is a need for effective blood-based screens for early-stage cancer based on circulating analytes.

The detection of circulating tumor DNA is increasingly acknowledged as a viable “liquid biopsy” approach, allowing for the detection and informative investigation of tumors in a non-invasive manner. In some cases, using the identification of tumor specific mutations, these techniques have been applied to colon, breast and prostate cancers. Due to the high background of normal (e.g., non-tumor-derived) DNA present in the circulation, these techniques may be limited in sensitivity.

The detection of tumor-specific TCR expression in the blood may offer distinct advantages over the detection of mutations. A number of single or multiple TCR sequences biomarkers may be assessed in cancers including but not limited to lung, colon, and breast.

There remains a need for more sensitive and specific screening tools for detecting early-stage or low tumor-burden cancer tumor signals in relapse and primary screening in at risk populations.

The present disclosure provides methods and systems directed to T-cell receptor expression profiling associated with cancer detection and disease progression.

In an aspect, the present disclosure provides methods that use a panel of T-cell receptor regions useful for the analysis of T-cell receptor expression within a region or gene, other aspects provide novel uses of the region, gene and the gene product as well as methods, assays and kits directed to detecting, differentiating and distinguishing cell proliferative disorders. The method and nucleic acids provided herein may be used for the analysis of cell proliferative disorders selected from the group consisting of adenocarcinomas, adenomas, polyps, squamous cell cancers, carcinoid tumors, sarcomas, and lymphomas.

### I. Definitions

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject”, generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject can be a person that has cancer or is suspected of having cancer. The subject may be displaying a symptom(s) indicative of a health or physiological state or condition of the subject, such as a cancer or other disease, disorder, or condition of the subject. As an alternative, the subject can be asymptomatic with respect to such health or physiological state or condition.

As used herein, the term “sample” generally refers to a biological sample obtained from or derived from one or more subjects. Biological samples may be tissue biopsies, stool specimens, blood samples, or cellular fractions of blood samples such as peripheral blood mononuclear cells (PBMCs). Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck tube), or a cell-free DNA collection tube (e.g., Streck). Cell-free biological samples may be derived from whole blood samples by fractionation. Biological samples or derivatives thereof may contain cells. For example, a biological sample may be a blood sample or a derivative thereof (e.g., blood collected by a collection tube or blood drops).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent

As used herein, the term “target nucleic acid” generally refers to a nucleic acid molecule in a starting population of nucleic acid molecules having a nucleotide sequence whose presence, amount, and/or sequence, or changes in one or more of these, are to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase

The term “cell-free nucleic acid (cfNA)”, as used herein, generally refers to nucleic acids (such as cell-free RNA (“cfRNA”) or cell-free DNA (“cfDNA”)) in a biological sample that are not contained in a cell. cfDNA may circulate freely in in a bodily fluid, such as in the bloodstream.

The term “cell-free sample”, as used herein, generally refers to a biological sample that is substantially devoid of intact cells. This may be derived from a biological sample that is itself substantially devoid of cells or may be derived from a sample from which cells have been removed. Examples of cell-free samples include those derived from blood, such as serum or plasma; urine; or samples derived from other sources, such as semen, sputum, feces, ductal exudate, lymph, or recovered lavage.

The term “circulating tumor DNA”, as used herein, generally refers to cfDNA originating from a tumor.

The term “genomic region”, as used herein, generally refers to identified regions of nucleic acid that are identified by their location in the chromosome. In some examples, the genomic regions are referred to by a gene name and encompass coding and non-coding regions associated with that physical region of nucleic acid. As used herein, a gene comprises coding regions (exons), non-coding regions (introns), transcriptional control or other regulatory regions, and promoters. In another example, the genomic region may incorporate an intron or exon or an intron/exon boundary within a named gene.

The term “cell proliferative disorder”, as used herein, generally refers to a disorder or disease that comprises disordered or aberrant proliferation of cells in an individual. In some examples, the disorder is selected from acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloepithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney′ cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom macroglobulinemia, and Wilms Tumor.

The terms cancer “type” and “subtype” generally are used relatively herein, such that one “type” of cancer, such as breast cancer, may be “subtypes” based on e.g., stage, morphology, histology, gene expression, receptor profile, mutation profile, aggressiveness, prognosis, malignant characteristics, etc. Likewise, “type” and “subtype” may be applied at a finer level, e.g., to differentiate one histological “type” into “subtypes”, e.g., defined according to mutation profile or gene expression. Cancer “stage” is also used to refer to classification of cancer types based on histological and pathological characteristics relating to disease progression.

### II. Assaying Samples

In certain aspects using biological samples, these biological samples may be obtained or derived from a human subject. Biological samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, cell-free RNA collection tubes, or cell-free DNA collection tubes).

The biological sample may be obtained from a subject with a cancer, from a subject that is suspected of having a cancer, from a subject that does not have or is not suspected of having the cancer, or from a subject exhibiting at least one sign or symptom of the cancer.

The biological sample may be taken before or after treatment of a subject with the cancer. Biological samples may be obtained from a subject during a treatment or a treatment regime. Multiple biological samples may be obtained from a subject to monitor the effects of the treatment over time. The biological sample may be taken from a subject having or suspected of having a cancer for which a definitive positive or negative diagnosis is not available via clinical tests. The sample may be taken from a subject suspected of having a cancer. The biological sample may be taken from a subject experiencing unexplained symptoms, such as fatigue, nausea, weight loss, aches and pains, weakness, or bleeding. The biological sample may be taken from a subject having explained symptoms. The biological sample may be taken from a subject at risk of developing a cancer due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In one embodiment, the biological sample is selected from a sample of cell-free biological sample (such as cell-free nucleic acid sample), plasma, serum, buffy coat, single cell or tissue.

A cell-free biological sample may contain one or more analytes capable of being assayed, such as cell-free ribonucleic acid (cfRNA) molecules suitable for assaying to generate transcriptomic data, cell-free deoxyribonucleic acid (cfDNA) molecules suitable for assaying to generate genomic data, or a mixture or combination thereof. One or more such analytes (e.g., cfRNA molecules and/or cfDNA molecules) may be isolated or extracted from one or more cell-free biological samples of a subject for downstream assaying using one or more suitable assays.

After obtaining a biological sample from the subject, the biological sample may be processed to generate datasets indicative of a cancer of the subject. For example, a presence, absence, or quantitative assessment of nucleic acid molecules of the cell-free biological sample at a panel of cancer-associated CDR3 sequences (e.g., quantitative measures of RNA transcripts or DNA at the cancer-associated genomic loci). Processing the cell-free biological sample obtained from the subject may comprise (i) subjecting the cell-free biological sample to conditions that are sufficient to isolate, enrich, or extract a plurality of nucleic acid molecules, and (ii) assaying the plurality of nucleic acid molecules to generate the dataset.

In some embodiments, a plurality of nucleic acid molecules is extracted from the biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a particular input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with cancers. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a biological sample may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the cancer. For example, the sequence reads may be aligned to one or more reference genomes (e.g., a genome of one or more species such as a human genome). The aligned sequence reads may be quantified at one or more genomic loci to generate the datasets indicative of the cancer. For example, quantification of sequences corresponding to a plurality of genomic loci associated with cancers may generate the datasets indicative of the cancer.

The cell-free biological sample may be processed without any nucleic acid extraction. For example, the cancer may be identified or monitored in the subject by using probes configured to selectively enrich nucleic acid (e.g., RNA or DNA) molecules corresponding to the plurality of T cell receptor or B cell receptor sequences. The probes may be nucleic acid primers. The probes may have sequence complementarity with nucleic acid sequences from one or more of the plurality of cancer-associated T cell receptor or B cell receptor sequences. The plurality of cancer-associated T cell receptor or B cell receptor sequences may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct cancer-associated T cell receptor or B cell receptor sequences.

The probes may be nucleic acid molecules (e.g., RNA or DNA) having sequence complementarity with nucleic acid sequences (e.g., RNA or DNA) of the one or more T cell receptor or B cell receptor sequences (e.g., cancer-associated T cell receptor or B cell receptor sequences). These nucleic acid molecules may be primers or comprise enrichment sequences. The assaying of the cell-free biological sample using probes that are selective for the one or more T cell receptor or B cell receptor sequences (e.g., cancer-associated T cell receptor or B cell receptor sequences) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more T cell receptor or B cell receptor sequences (e.g., cancer-associated T cell receptor or B cell receptor sequences) to generate the data indicative of the cancer. For example, quantification of array hybridization or polymerase chain reaction (PCR) corresponding to a plurality of T cell receptor or B cell receptor sequences (e.g., cancer-associated T cell receptor or B cell receptor sequences) may generate data indicative of the cancer. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays may be used to simultaneously process cell-free biological samples of a subject. For example, a first assay may be used to process a first cell-free biological sample obtained or derived from the subject to generate a first dataset indicative of the cancer; and a second assay different from the first assay may be used to process a second cell-free biological sample obtained or derived from the subject to generate a second dataset indicative of the cancer. Any or all of the first dataset and the second dataset may then be analyzed to assess the cancer of the subject. For example, a single diagnostic index or diagnosis score can be generated based on a combination of the first dataset and the second dataset. As another example, separate diagnostic indexes or diagnosis scores can be generated based on the first dataset and the second dataset.

In certain embodiments, the cell-free biological samples may be processed using a methylation-specific assay. For example, a methylation-specific assay can be used to identify a quantitative measure (e.g., indicative of a presence, absence, or relative amount) of methylation each of a plurality of cancer-associated T cell receptor or B cell receptor sequences in a cell-free biological sample of the subject. The methylation-specific assay may be configured to process cell-free biological samples such as a blood sample or a urine sample (or derivatives thereof) of the subject. A quantitative measure (e.g., indicative of a presence, absence, or relative amount) of methylation of cancer-associated T cell receptor or B cell receptor sequences in the cell-free biological sample may be indicative of one or more cancers. The methylation-specific assay may be used to generate datasets indicative of the quantitative measure (e.g., indicative of a presence, absence, or relative amount) of methylation of each of a plurality of cancer-associated T cell receptor or B cell receptor sequences in the cell-free biological sample of the subject.

The methylation-specific assay may comprise, for example, one or more of: a methylation-aware sequencing (e.g., using bisulfite treatment), enzymatic methylation-specific sequencing, pyrosequencing, methylation-sensitive single-strand conformation analysis (MS-SSCA), high-resolution melting analysis (HRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

### III. T-Cell Receptor and B-Cell Receptor CDR3 Sequence Analysis

The present disclosure provides methods and systems to analyze biological samples to obtain measurable features from T-cell receptor and/or B-cell receptor sequences in the sample that are associated with the development of cell proliferative disorders. The features from the sequence data obtained spanning the CDR3 regions in a biological sample may be processed using a trained algorithm (e.g., a machine learning model) to create a classifier configured to stratify a population of individuals with a cell proliferative disorder.

In various embodiments, standard sequencing methods may be used to obtain sequence information spanning the CDR3 region. In certain embodiments, substantially all of the CDR3 sequences in the biological sample can be sequenced. In certain embodiments, separation of non-relevant or noisy sequences can be performed during the analysis after sequencing to increase the relative amount of sequences contributing to the signal of the particular biological state being interrogated or classified in the sample.

Since the CDR3 region exhibits high variability, probes and primers that are complementary to sequences outside, but still proximal to, this region are used to facilitate obtaining sequence information of CDR3 regions present in the biological sample. Oligonucleotide primers complementary to the relatively more constant sequence regions that flank the CDR3 region are used to direct sequencing of this region as primers for PCR-based sequencing approaches. In various embodiments, oligonucleotides complementary to the V and J regions are used as primers for amplicon-based methods. In other embodiments, oligonucleotide primers that are complementary to the relatively more constant sequence regions that flank the CDR3 region are used to direct sequencing of this region as probes for target-capture enrichment approaches prior to sequencing. In various embodiments, oligonucleotides complementary to the V and J regions are used as probes for target-enrichment methods.

The average CDR3 length is ˜50 nucleotides. V(D)J recombination can entail 52 V segments and 6 or 7 J segments spanning ˜10 kb of genomic sequence. In certain embodiments, oligonucleotides are complementary to the V segment and are located within 50 nt, 100 nt, 150 nt, 200 nt, 300 nt, or 400 nt of the V-D junction. In other embodiments, oligonucleotides span the J segment.

In certain embodiments that employ methylated sequence analysis, these oligonucleotides are modified accordingly with standard methods to be used after nucleic acid conversion operations used in methylation sequencing.

In some embodiments, the designing of the plurality of primer pairs comprising converting non-methylated cytosines uracil, to simulate cytosine to uracil conversion, and designing the primer pairs using the converted sequence.

In some embodiments, the primer pairs are designed to have a methylation bias.

In some embodiments, the primer pairs are methylation-specific.

In some embodiments, the primer pairs have no CpG bases within them having utility for methylation-specific or non-methylation-specific sequencing.

In another aspect the probes and/or primers are preselected based on CapTCR (Mulder et al. Blood Adv. 2018)

In one embodiment, the probes and/or primers are preselected to hybridize to substantially all α, β, γ, δ loci.

In one embodiment, the probes and/or primers are preselected to represent substantially complete population diversity by incorporating all unique V/J combinations explicit in the IMGT database.

In one embodiment, the probes and/or primers are preselected to represent the α locus and extended by 20 nucleotides (nt).

In one embodiment, the probes and/or primers are preselected to represent the β locus and extended by nt.

In one embodiment, the probes and/or primers are preselected to represent the γ locus and extended by nt.

In one embodiment, the probes and/or primers are preselected to represent the δ locus and extended by nt.

In one embodiment, the probes and/or primers are preselected to represent the β locus and extended by nt.

T cell receptor or B cell receptor sequences can be amplified from nucleic acid in a multiplex reaction using at least one primer that anneals to the J region and one or more primers that can anneal to one or more V segments. The number of primers that anneal to V segments in a multiplex reaction can be, for example, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80. The number of primers that anneal to V segments in a multiplex reaction can be, for example, 10-60, 20-50, 30-50, 40-50, 20-40, 30-40, or 35-40. The primers can anneal to different V segments.

The region to be sequenced can include the full clonal sequence or a subset of the clonal sequence, including the V-D junction, D-J junction of an immunoglobulin or T-cell receptor gene, the full variable region of an immunoglobulin or T-cell receptor gene, the antigen recognition region, or a CDR, e.g., complementarity determining region 3 (CDR3).

The CDR3 sequence can amplified using a primary and a secondary amplification operation. Each of the different amplification operations can comprise different primers. The different primers can introduce sequence not original present in the immune gene sequence. For example, the amplification procedure can add one or more tags to 5′ and/or 3′ end of amplified CDR3 sequence. The tag can be sequence that facilitates subsequent sequencing of the amplified DNA. The tag can be sequence that facilitates binding the amplified sequence to a solid support.

Other methods for amplification may not employ any primers in the V region. Instead, a specific primer can be used from the J segment and a generic primer can be put in the other side (5′). The generic primer can be appended in the cDNA synthesis through different methods including the well described methods of strand switching. Similarly, the generic primer can be appended after cDNA making through different methods including ligation.

In certain embodiments, sequencing can be performed with TCRseq amplicon-based sequencing protocol (Adaptive Biotech, Seattle, WA). Sequencing data can be generated from amplicon-based library methods from either whole blood, plasma, serum or sorted PBMCs.

The average CDR3 length is ˜50 nucleotides and encodes 12-17 amino acid residues. V(D)J recombination provides the biological variation required and therefore hybridization probes are used to target all V segments (n=52) and J segments (n=6 or 7) thereby sequencing ˜20 kb of genomic sequence. In certain embodiments, the probe sequence can be adjusted to reflect enzymatic conversion (methylated and unmethylated probes).

Use a pre-conversion plasma aliquot and a dedicated assay that may provide higher TCR sequence yield and full sequence complexity.

In one embodiment, the biological sample is buffy coat isolated from a blood sample. In some embodiments, a TCR assay is performed directly on PBMCs isolated from the buffy coat. In certain embodiments, the TCR assay includes for example amplicon-based TCR assays.

In certain embodiments, the T cell receptor or B cell receptor sequences are featurized and the TCR assay results are featurized and both sets of features are used in a machine learning model to characterize a sample based on T cell receptor or B cell receptor repertoire.

In various embodiments, characteristics of CDR3 regions selected from hydrophobicity, secondary structure, size/mass, codon degeneracy or electric charge are be used as features in machine learning models.

### Targeted Sequencing

In targeted sequencing approaches, targeted regions in a biological sample such as cfDNA are analyzed in order to sequence genomic regions of particular biological importance. In some embodiments, the target region comprises, or hybridizes under stringent conditions to, contiguous nucleotides of target regions of interest, such as at least about 16 contiguous nucleotides of a target region of interest. In different examples, targeted sequencing may be accomplished using hybridization capture and amplicon sequencing approaches.

### Hybridization Capture

The hybridization method provided herein may be used in various formats of nucleic acid hybridizations, such as in-solution hybridization and such as hybridization on a solid support (e.g., Northern, Southern and in situ hybridization on membranes, microarrays and cell/tissue slides). In particular, the method can be suitable for in-solution hybrid capture for target enrichment of certain types of genomic DNA sequences (e.g., exons) employed in targeted next-generation sequencing. For hybrid capture approaches, a cell-free nucleic acid sample can be subjected to library preparation. As used herein, “library preparation” comprises end-repair, A-tailing, adapter ligation, or any other preparation performed on the cell-free DNA to permit subsequent sequencing of DNA. In certain examples, a prepared cell-free nucleic acid library sequence contains adapters, sequence tags, index barcodes that are ligated onto cell-free nucleic acid sample molecules. Various commercially available kits are available to facilitate library preparation for next-generation sequencing approaches. Next-generation sequencing library construction may comprise preparing nucleic acids targets using a coordinated series of enzymatic reactions to produce a random collection of DNA fragments, of specific size, for high throughput sequencing. Advances and the development of various library preparation technologies have expanded the application of next-generation sequencing to fields such as transcriptomics and epigenetics.

Improvements in sequencing technologies have resulted in changes and improvements to library preparation. Next-generation sequencing library preparation kits, developed by companies such as Agilent, Bioo Scientific, Kapa Biosystems, New England Biolabs, Illumina, Life Technologies, Pacific Biosciences and Roche provide consistency and reproducibility to various molecular biology reactions that ensure compatibility with the latest NGS instrument technology.

In various examples for targeted capture gene panels, various library preparation kits may be selected from Nextera Flex (Illumina), IonAmpliseq (Thermo Fisher Scientific), and Genexus (Thermo Fisher Scientific), Agilent ClearSeq (Illumina), Agilent SureSelect Capture (Illumina), Archer FusionPlex (Illumina), BiooScientific NEXTflex (Illumina), IDT xGen (Illumina), Illumina TruSight (Illumina), Nimblegene SeqCap (Illumina), and Qiagen GeneRead (Illumina).

In some embodiments, the hybrid capture method is carried out on the prepared library sequences using specific probes. In some embodiments, the term “specific probe”, as used herein, generally refers to a probe that is specific for particular defined methylation sites. In some embodiments, the specific probes are designed based on using human genome as a reference sequence and using specified genomic regions predicted or validated to have methylation sites as target sequences. Specifically, the genomic regions predicted or validated to have methylation sites may comprise at least one of the following: a promoter region, a CpG island region, a CGI shore region, and a imprinted gene region. Therefore, when carrying out the hybrid capture by using the specific probes of some embodiments, the sequences in the sample genome which are complimentary to the target sequences, e.g., regions in the sample genome predicted or validated to have methylation sites (which are also referred to as “specified genomic regions” herein) may be captured efficiently.

According to an example, the methylated regions described herein are used for designing the specific probes. In some embodiments, the specific probes are designed using commercially available methods such as for example an eArray system. The length of the probes may be sufficient to hybridize with sufficient specificity to the methylated region of interest. In various embodiments, the probe is a 10-mer, 11-mer, 12-mer, 13-mer, 14-mer 15-mer, 16-mer, 17-mer, 18-mer, 19-mer, or 20-mer.

### Amplicon-Based Sequencing

In-house data can be generated from standard amplicon-based library methods from either whole blood or sorted PBMCs. Fragments of the DNA may be amplified. In some cases for methylation analysis, the amplifying can be carried out with primers designed to anneal to methylation converted target sequences having at least one methylated site therein. Methylation sequencing conversion results in unmethylated cytosines being converted to uracil, while 5-methylcytosine is unaffected. “Converted target sequences” are thus understood to be sequences in which cytosines predicted or validated to be methylation sites are fixed as “C” (cytosine), while cytosines predicted or validated to be unmethylated are fixed as “U” (uracil; which may be treated as “T” (thymine) for primer design purposes).

In various examples, the source of the DNA can be cell-free DNA from whole blood, plasma, serum, or genomic DNA extracted from cells or tissue. In some embodiments, the size of the amplified fragment is between about 100 and 200 base pairs in length. In some embodiments, the DNA source is extracted from cellular sources (e.g., tissues, biopsies, cell lines), and the amplified fragment is between about 100 and 350 base pairs in length. In some embodiments, the amplified fragment comprises at least one 20 base pair sequence comprising at least one, at least two, at least three, or more than three CpG dinucleotides. The amplification may be carried out using sets of primer oligonucleotides according to the present disclosure, and may use a heat-stable polymerase. The amplification of several DNA segments may be carried out simultaneously in one and the same reaction vessel, In some embodiments of the method, two or more fragments are amplified simultaneously. For example, the amplification may be carried out using a polymerase chain reaction (PCR).

Primers designed to target such sequences may exhibit a degree of bias towards converted methylated sequences. In some embodiments, the PCR primers are designed to be methylation specific for targeted methylation-sequencing applications. This may allow for greater sensitivity in some applications. For instance, primers may be designed to include a discriminatory nucleotide (specific to a methylated sequence following bisulfite conversion) positioned to achieve optimal discrimination, e.g., in PCR applications. The discriminatory may be positioned at the 3′ ultimate or penultimate position.

In some embodiments, the primers are designed to amplify DNA fragments 75 to 350 nucleotides in length. This is the consensus size range for circulating DNA and optimizing primer design to account for target size may increase the sensitivity of the method according to this example. The primers may be designed to amplify regions that are about 50 to 200, about 75 to 150, or about 100 or 125 nucleotides in length containing portions or all of the CDR3 segment.

### IV. Classifiers, Machine Learning Models & Systems

In various examples, TCR sequencing features are used as input datasets into trained algorithms (e.g., machine learning models or classifiers) to find correlations between sequence composition and patient groups. Examples of such patient groups include presence of diseases or conditions, stages, subtypes, responders vs. non-responders, and progressors vs. non-progressors. In various examples, feature matrices are generated to compare samples obtained from individuals with defined conditions or characteristics. In some embodiments, samples are obtained from healthy individuals, or individuals who do not have any of the defined indications and samples from patients having or exhibiting symptoms of cancer.

In some cases, the samples from which the T cell receptor or B cell receptor sequences are obtained are associated with the presence of a biological trait which can be used to train the machine learning model.

In some embodiments, the biological trait comprises malignancy.

In some embodiments, the biological trait comprises a cancer type.

In some embodiments, the biological trait comprises a cancer stage.

In some embodiments, the biological trait comprises a cancer classification.

In some embodiments, the cancer classification comprises a cancer grade.

In some embodiments, the cancer classification comprises a histological classification.

In some embodiments, the biological trait comprises a metabolic profile.

In some embodiments, the biological trait comprises a mutation.

In some embodiments, the mutation is a disease-associated mutation.

In some embodiments, the biological trait comprises a clinical outcome.

In some embodiments, the biological trait comprises a drug response.

In certain embodiments, methods to analyze TCR and BCR sequence information may include TRUST (Li et al., Nature Genetics, 2017), Deep-TCR (Sidhom et al 2018), DeepCAT (Beshnova et al 2020), TCRex (Gielis et al. 2018), TCRdist (Dash et al. 2017), NetTCR (Jurtz et al. 2018), TCRGP (Jokinen et al. 2019), TCRNET (Pogorelyy et al. 2019), or IGOR (Marcou et al. 2018).

In certain embodiments, BCR and TCR repertoire sequence information from cfDNA samples is analyzed with the TCR/BCR Receptor Utilities for Solid Tumors (TRUST) software which was originally designed for solid tumors. TRUST extracts T/B cell receptor hypervariable CDR3 sequences from unselected tumor RNA-seq data. It is an ultra-sensitive de novo assembly method for calling CDR3s (Li et al., Nature Genetics, 2017), with demonstrated utilities when applied to large cancer genomics data (Li et al., Nature Genetics, 2016).

In some embodiments, sequence information from cfDNA samples is analyzed by computational analysis.

In one embodiment, the cfDNA sequence information is analyzed with the Deep-TCR software which is a broad collection of unsupervised and supervised deep learning methods able to uncover structure in highly complex and large TCR sequencing data.

In certain embodiments, sequence information from cfDNA samples is analyzed with the Deep CNN Model for Cancer Associated TCRs (DeepCAT) software. DeepCAT is a computational method based on convolutional neural network to exclusively identify cancer-associated beta chain TCR hypervariable CDR3 sequences (Li et al., Science Translational Medicine, 2020). In various embodiments, DeepCAT is employed for analyzing T cell receptor and/or B cell receptor sequences and can be used in a length-agnostic/independent model.

In one embodiment, the computational analysis comprises removing non-CDR3 sequence information from the sequence data set.

In one embodiment, the computational analysis comprises DNA sequence alignment, assembly, and featurization, PCA, MiXCR, CNN, Deep-TCR, or DeepCAT methods on T cell and/or B cell receptor sequences.

As used herein, as it relates to machine learning and pattern recognition the term “feature” generally refers to an individual measurable property or characteristic of a phenomenon being observed. The concept of “feature” is related to that of explanatory variable used in statistical techniques such as for example, but not limited to, linear regression and logistic regression. Features are usually numeric, but structural features such as strings and graphs are used in syntactic pattern recognition.

The term “input features” (or “features”), as used herein, generally refers to variables that are used by the trained algorithm (e.g., model or classifier) to predict an output classification (label) of a sample, e.g., a condition, sequence content (e.g., mutations), suggested data collection operations, or suggested treatments. Values of the variables may be determined for a sample and used to determine a classification.

In certain embodiments, input features for TCR and/or BCR analysis include but are not limited to clonality, abundance and frequency metrics, primary amino acid sequences/strings, and/or latent representations of biophysical and chemical properties of amino acid sequences.

For a plurality of assays, the system identifies feature sets to input into a trained algorithm (e.g., machine learning model or classifier). The system performs an assay on each molecule class and forms a feature vector from the measured values. The system inputs the feature vector into the machine learning model and obtains an output classification of whether the biological sample has a specified property.

In various embodiments, immune-derived biological signals in genomic or cfDNA can be represented as numerical values characteristic of cellular composition (immune cell type of origin for sequence fragments), genes and biological pathways they involve, transcription factor activity (such as transcription factor binding, silencing, or activation).

In various embodiments, immune-derived biological signals in genomic or cfDNA can be represented as numerical values characterizing T cell receptor and/or B cell receptor repertoire such as repertoire diversity, infiltration or clonal expansion, somatic hypermutation or isotype class switch (for example switching between IgA, IgG, IgG3-1.

In some embodiments, the machine learning model outputs a classifier capable of distinguishing between two or more groups or classes of individuals or features in a population of individuals or features of the population. In some embodiments, the classifier is a trained machine learning classifier.

In some embodiments, the informative loci or features of biomarkers in a cancer tissue are assayed to form a profile. Receiver-operating characteristic (ROC) curves may be generated by plotting the performance of a particular feature (e.g., any of the biomarkers described herein and/or any item of additional biomedical information) in distinguishing between two populations (e.g., individuals responding and not responding to a therapeutic agent). In some embodiments, the feature data across the entire population (e.g., the cases and controls) are sorted in ascending order based on the value of a single feature.

In various embodiments, the specified property is selected from healthy vs. cancer, disease subtype, disease stage, progressor vs. non-progressor, and responder vs. non-responder.

### A. Data Analysis

In some examples, the present disclosure provides a system, method, or kit having data analysis realized in software application, computing hardware, or both. In various examples, the analysis application or system comprises at least a data receiving module, a data pre-processing module, a data analysis module (which can operate on one or more types of genomic data), a data interpretation module, or a data visualization module. In some embodiments, the data receiving module can comprise computer systems that connect laboratory hardware or instrumentation with computer systems that process laboratory data. In some embodiments, the data pre-processing module can comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

In various examples, machine learning methods are applied to distinguish samples in a population of samples. In some embodiments, machine learning methods are applied to distinguish samples between healthy and advanced disease (e.g., adenoma) samples.

In some embodiments, the one or more machine learning operations used to train the prediction engine include one or more of: a generalized linear model, a generalized additive model, a non-parametric regression operation, a random forest classifier, a spatial regression operation, a Bayesian regression model, a time series analysis, a Bayesian network, a Gaussian network, a decision tree learning operation, an artificial neural network, a recurrent neural network, a convolutional neural network, a reinforcement learning operation, linear or non-linear regression operations, a support vector machine, a clustering operation, and a genetic algorithm operation.

In various examples, computer processing methods are selected from logistic regression, multiple linear regression (MLR), dimension reduction, partial least squares (PLS) regression, principal component regression, autoencoders, variational autoencoders, singular value decomposition, generative adversarial networks, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, and artificial neural networks.

In some examples, the methods disclosed herein can include computational analysis on nucleic acid sequencing data of samples from an individual or from a plurality of individuals.

### B. Classifier Generation

In an aspect, the disclosed systems and methods provide a classifier generated based on feature information derived from methylation sequence analysis from biological samples of cfDNA. The classifier forms part of a predictive engine for distinguishing groups in a population based on sequence features identified in biological samples such as cfDNA

In some embodiments, a classifier is created by normalizing the sequence information by formatting similar portions of the sequence information into a unified format and a unified scale; storing the normalized sequence information in a columnar database; training a prediction engine by applying one or more one machine learning operations to the stored normalized sequence information, the prediction engine mapping, for a particular population, a combination of one or more features; applying the prediction engine to the accessed field information to identify an individual associated with a group; and classifying the individual into a group.

Specificity, as used herein, generally refers to “the probability of a negative test among those who are free from the disease”. It may be calculated by the number of disease-free persons who tested negative divided by the total number of disease-free individuals.

In various examples, the model, classifier, or predictive test has a specificity of at least 40%, at least 45%, at least 50%, at least 55%, at least 60%, at least 65%, at least 70%, at least 75%, at least 80%, at least 85%, at least 90%, at least 95%, or at least 99%.

Sensitivity, as used herein, generally refers to “the probability of a positive test among those who have the disease”. It may be calculated by the number of diseased individuals who tested positive divided by the total number of diseased individuals.

In various examples, the model, classifier, or predictive test has a sensitivity of at least 40%, at least 45%, at least 50%, at least 55%, at least 60%, at least 65%, at least 70%, at least 75%, at least 80%, at least 85%, at least 90%, at least 95%, or at least 99%.

### C. Digital Processing Device

In some examples, the subject matter described herein can include a digital processing device or use of the same. In some examples, the digital processing device can include one or more hardware central processing units (CPU), graphics processing units (GPU), or tensor processing units (TPU) that carry out the device's functions. In some examples, the digital processing device can include an operating system configured to perform executable instructions.

In some examples, the digital processing device can optionally be connected a computer network. In some examples, the digital processing device may be optionally connected to the Internet. In some examples, the digital processing device may be optionally connected to a cloud computing infrastructure. In some examples, the digital processing device may be optionally connected to an intranet. In some examples, the digital processing device may be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations.

In some examples, the digital processing device can include an operating system configured to perform executable instructions. For example, the operating system can include software, including programs and data, which manages the device's hardware and provides services for execution of applications. Non-limiting examples of operating systems include Ubuntu, FreeBSD, OpenBSD, NetBSD®, Linux, Apple® Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some examples, the operating system may be provided by cloud computing, and cloud computing resources may be provided by one or more service providers.

In some examples, the device can include a storage and/or memory device. The storage and/or memory device may be one or more physical apparatuses used to store data or programs on a temporary or permanent basis. In some examples, the device may be volatile memory and require power to maintain stored information. In some examples, the device may be non-volatile memory and retain stored information when the digital processing device is not powered. In some examples, the non-volatile memory can include flash memory. In some examples, the non-volatile memory can include dynamic random-access memory (DRAM). In some examples, the non-volatile memory can include ferroelectric random-access memory (FRAM). In some examples, the non-volatile memory can include phase-change random access memory (PRAM).

In some examples, the device may be a storage device including, for example, CD-ROMs, DVDs, flash memory devices, magnetic disk drives, magnetic tapes drives, optical disk drives, and cloud computing-based storage. In some examples, the storage and/or memory device may be a combination of devices such as those disclosed herein. In some examples, the digital processing device can include a display to send visual information to a user. In some examples, the display may be a cathode ray tube (CRT). In some examples, the display may be a liquid crystal display (LCD). In some examples, the display may be a thin film transistor liquid crystal display (TFT-LCD). In some examples, the display may be an organic light emitting diode (OLED) display. In some examples, on OLED display may be a passive-matrix OLED (PMOLED) or active-matrix OLED (AMOLED) display. In some examples, the display may be a plasma display. In some examples, the display may be a video projector. In some examples, the display may be a combination of devices such as those disclosed herein.

In some examples, the digital processing device can include an input device to receive information from a user. In some examples, the input device may be a keyboard. In some examples, the input device may be a pointing device including, for example, a mouse, trackball, track pad, joystick, game controller, or stylus. In some examples, the input device may be a touch screen or a multi-touch screen. In some examples, the input device may be a microphone to capture voice or other sound input. In some examples, the input device may be a video camera to capture motion or visual input. In some examples, the input device may be a combination of devices such as those disclosed herein.

### D. Non-Transitory Computer-Readable Storage Medium

In some examples, the subject matter disclosed herein can include one or more non-transitory computer-readable storage media encoded with a program including instructions executable by the operating system of an optionally networked digital processing device. In some examples, a computer-readable storage medium may be a tangible component of a digital processing device. In some examples, a computer-readable storage medium may be optionally removable from a digital processing device. In some examples, a computer-readable storage medium can include, for example, CD-ROMs, DVDs, flash memory devices, solid state memory, magnetic disk drives, magnetic tape drives, optical disk drives, cloud computing systems and services, and the like. In some examples, the program and instructions may be permanently, substantially permanently, semi-permanently, or non-transitorily encoded on the media.

### E. Computer Systems

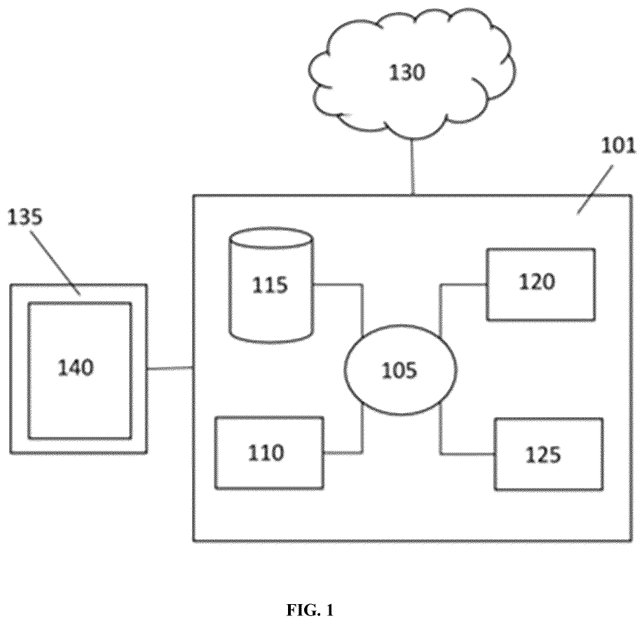

The present disclosure provides computer systems that are programmed to implement methods described herein. FIG. 1 shows a computer system 101 that is programmed or otherwise configured to store, process, identify, or interpret patient data, biological data, biological sequences, and reference sequences. The computer system 101 can process various aspects of patient data, biological data, biological sequences, or reference sequences of the present disclosure (FIG. 1). The computer system 101 may be an electronic device of a user or a computer system that is remotely located with respect to the electronic device. The electronic device may be a mobile electronic device.

The computer system 101 comprises a central processing unit (CPU, also “processor” and “computer processor” herein) 105, which may be a single core or multi core processor, or a plurality of processors for parallel processing. The computer system 101 also comprises memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 may be a data storage unit (or data repository) for storing data. The computer system 101 may be operatively coupled to a computer network (“network”) 130 with the aid of the communication interface 120. The network 130 may be the Internet, an internet and/or extranet, or an intranet and/or extranet that is in communication with the Internet. The network 130 in some examples is a telecommunication and/or data network. The network 130 can include one or more computer servers, which can enable distributed computing, such as cloud computing. The network 130, in some examples with the aid of the computer system 101, can implement a peer-to-peer network, which may enable devices coupled to the computer system 101 to behave as a client or a server.

The CPU 105 can execute a sequence of machine-readable instructions, which may be embodied in a program or software. The instructions may be stored in a memory location, such as the memory 110. The instructions may be directed to the CPU 105, which can subsequently program or otherwise configure the CPU 105 to implement methods of the present disclosure. Examples of operations performed by the CPU 105 can include fetch, decode, execute, and writeback.

The CPU 105 may be part of a circuit, such as an integrated circuit. One or more other components of the system 101 may be included in the circuit. In some examples, the circuit is an application specific integrated circuit (ASIC).

The storage unit 115 can store files, such as drivers, libraries and saved programs. The storage unit 115 can store user data, e.g., user preferences and user programs. The computer system 101 in some examples can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130.

Methods as described herein may be implemented by way of machine (e.g., computer processor) executable code stored on an electronic storage location of the computer system 101, such as, for example, on the memory 110 or electronic storage unit 115. The machine-executable or machine-readable code may be provided in the form of software. During use, the code may be executed by the processor 105. In some examples, the code may be retrieved from the storage unit 115 and stored on the memory 110 for ready access by the processor 105. In some examples, the electronic storage unit 115 may be precluded, and machine-executable instructions are stored on memory 110.

The code may be pre-compiled and configured for use with a machine having a processer adapted to execute the code or may be interpreted or compiled during runtime. The code may be supplied in a programming language that may be selected to enable the code to execute in a pre-compiled, interpreted, or as-compiled fashion.

Aspects of the systems and methods provided herein, such as the computer system 101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements comprises optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a nucleic acid sequence, an enriched nucleic acid sample, a methylation profile, an expression profile, and an analysis of a methylation or expression profile. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 105. The algorithm can, for example, store, process, identify, or interpret patient data, biological data, biological sequences, and reference sequences.

While certain examples of methods and systems have been shown and described herein, one of skill in the art will realize that these are provided by way of example only and not intended to be limiting within the specification. Numerous variations, changes, and substitutions will now occur to those skilled in the art without departing from the scope described herein. Furthermore, it shall be understood that all aspects of the described methods and systems are not limited to the specific depictions, configurations or relative proportions set forth herein which depend upon a variety of conditions and variables and the description is intended to include such alternatives, modifications, variations or equivalents.

In some examples, the subject matter disclosed herein can include at least one computer program or use of the same. A computer program can a sequence of instructions, executable in the digital processing device's CPU, GPU, or TPU, written to perform a specified task. Computer-readable instructions may be implemented as program modules, such as functions, objects, Application Programming Interfaces (APIs), data structures, and the like, that perform particular tasks or implement particular abstract data types. In light of the disclosure provided herein, a computer program may be written in various versions of various languages.

The functionality of the computer-readable instructions may be combined or distributed in various environments. In some examples, a computer program can include one sequence of instructions. In some examples, a computer program can include a plurality of sequences of instructions. In some examples, a computer program may be provided from one location. In some examples, a computer program may be provided from a plurality of locations. In some examples, a computer program can include one or more software modules. In some examples, a computer program can include, in part or in whole, one or more web applications, one or more mobile applications, one or more standalone applications, one or more web browser plug-ins, extensions, add-ins, or add-ons, or combinations thereof.

In some examples, the computer processing may be a method of statistics, mathematics, biology, or any combination thereof. In some examples, the computer processing method comprises a dimension reduction method including, for example, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, and neural network such as convolutional neural networks.

In some embodiments, the computer processing method is a supervised machine learning method including, for example, a regression, support vector machine, tree-based method, and network. In supervised learning approaches, a group of samples from two or more groups are generally analyzed or processed with a statistical classification method. Sequence or expression level can be used as a basis for classifier that differentiates between the two or more groups. A new sample can then be analyzed or processed so that the classifier can associate the new sample with one of the two or more groups. Classification using supervised methods is generally performed by the following methodology:
1. Gather a training set. These can include, for example, sequence information from nucleic acid molecules sequenced herein.
2. Determine the input “feature” representation of the learned function. The accuracy of the learned function depends on how the input object is represented. Typically, the input object is transformed into a feature vector, which contains a number of features that are descriptive of the object.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g. classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set. The built model can involve feature coefficients or importance measures assigned to individual features.

Once the classifier (e.g. classification model) is determined as described above (“trained”), it can be used to classify a sample.

In some embodiments, the computer processing method is an unsupervised machine learning method including, for example, clustering, network, principal component analysis, and matrix factorization.

### F. Databases

In some examples, the subject matter disclosed herein can include one or more databases, or use of the same to store patient data, biological data, biological sequences, or reference sequences. Reference sequences may be derived from a database. In view of the disclosure provided herein, many databases may be suitable for storage and retrieval of the sequence information. In some examples, suitable databases can include, for example, relational databases, non-relational databases, object-oriented databases, object databases, entity-relationship model databases, associative databases, and XML databases. In some examples, a database may be internet-based. In some examples, a database may be web-based. In some examples, a database may be cloud computing-based. In some examples, a database may be based on one or more local computer storage devices.

In an aspect, the present disclosure provides a non-transitory computer-readable medium comprising instructions that direct a processor to carry out a method disclosed herein.

In an aspect, the present disclosure provides a computing device comprising the computer-readable medium.

In another aspect, the present disclosure provides a system for performing classifications of biological samples comprising:
a) a receiver to receive a plurality of training samples, each of the plurality of training samples having a plurality of classes of molecules, wherein each of the plurality of training samples comprises one or more defined labels;
b) a feature module to identify a set of features corresponding to an assay that are operable to be input to the machine learning model for each of the plurality of training samples, wherein the set of features correspond to properties of molecules in the plurality of training samples, wherein for each of the plurality of training samples, the system is operable to subject a plurality of classes of molecules in the training sample to a plurality of different assays to obtain sets of measured values, wherein each set of measured values is from one assay applied to a class of molecules in the training sample, wherein a plurality of sets of measured values are obtained for the plurality of training samples;
c) an analysis module to analyze the sets of measured values to obtain a training vector for the training sample, wherein the training vector comprises feature values of the N set of features of the corresponding assay, each feature value corresponding to a feature and including one or more measured values, wherein the training vector is formed using at least one feature from at least two of the N sets of features corresponding to a first subset of the plurality of different assays;
d) a labeling module to inform the system on the training vectors using parameters of the machine learning model to obtain output labels for the plurality of training samples;
e) a comparator module to compare the output labels to the defined labels of the training samples;
f) a training module to iteratively search for optimal values of the parameters as part of training the machine learning model based on the comparing the output labels to the defined labels of the training samples; and
g) an output module to provide the parameters of the machine learning model and the set of features for the machine learning model.

### V. Methods of Classifying Subjects in a Population

The disclosed methods are generally directed to ascertaining genetic and/or epigenetic parameters of genomic DNA associated with cell proliferative disorders via analysis of T cell repertoire and B cell repertoire in a subject. The method can be used in the improved diagnosis, treatment and monitoring of cell proliferative disorders, more specifically by enabling the improved identification of and differentiation between stages or subclasses of said disorders and the genetic predisposition to said disorders.

In certain embodiments, obtaining a profile of T cell repertoire or B cell repertoire in a subject is used to capture aspects of biology that are indicative of the presence of cell proliferative disorders or characteristics of cell proliferative disorders including but not limited to stage, tissue type, or treatment responsiveness. The T cell repertoire and/or B cell repertoire provides information on tumor infiltration, cell type diversity, and isotype switching (such as between IgA, IgG, IgG3-1) which is featurized and used in machine learning classification models such as those described herein.

Generally, the present disclosure provides a method for detecting a cell proliferative disorder that may be applied to cell-free samples, e.g., to detect cell-free circulating cell proliferative disorder DNA.

In some embodiments, the colon cell proliferative disorder is selected from acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloepithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney′ cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom macroglobulinemia, and Wilms Tumor, and any combination thereof.

In some embodiments, the cell proliferative disorder is a colon cell proliferative disorder is selected from adenoma (adenomatous polyps), sessile serrated adenoma (SSA), advanced adenoma, colorectal dysplasia, colorectal adenoma, colorectal cancer, colon cancer, rectal cancer, colorectal carcinoma, colorectal adenocarcinoma, carcinoid tumors, gastrointestinal carcinoid tumors, gastrointestinal stromal tumors (GISTs), lymphomas, and sarcomas, and any combination thereof.

In an aspect, the present disclosure provides a method for detecting a cell proliferative disorder, comprising: extracting DNA from a cell-free sample obtained from a subject, converting at least a portion of the DNA for methylation-specific sequencing, amplifying regions methylated in cancer from the converted DNA, generating sequencing reads from the amplified regions, and detecting cell proliferative disorder signals comprising at least one, at least two, at least three, or more than three methylated regions within a cancer panel, to obtain input features that are inputted into a machine learning model to obtain a classifier capable of discriminating between two groups of subjects (e.g., healthy vs cancer, disease stage, advanced adenoma vs cancer).

The trained machine learning methods, models, and discriminate classifiers described herein may be applied toward various medical applications including cancer detection, diagnosis and treatment responsiveness. As models may be trained with individual metadata and analyte-derived features, the applications may be tailored to stratify individuals in a population and guide treatment decisions accordingly.

### Diagnosis

Methods and systems provided herein may perform predictive analytics using artificial intelligence-based approaches to analyze acquired data from a subject (patient) to generate an output of diagnosis of the subject having a cell proliferative disorder such as cancer. For example, the application may apply a prediction algorithm to the acquired data to generate the diagnosis of the subject having the cancer. The prediction algorithm may comprise an artificial intelligence-based predictor, such as a machine learning-based predictor, configured to process the acquired data to generate the diagnosis of the subject having the cancer.

The machine learning predictor may be trained using datasets e.g., datasets generated by performing methylation assays using the signature panels described herein on biological samples of individuals from one or more sets of cohorts of patients having cancer as inputs and diagnosis (e.g., staging and/or tumor fraction) outcomes of the subjects as outputs to the machine learning predictor.

Training datasets (e.g., datasets generated by performing methylation assays using the signature panels described herein on biological samples of individuals) may be generated from, for example, one or more sets of subjects having common characteristics (features) and outcomes (labels). Training datasets may comprise a set of features and labels corresponding to the features relating to diagnosis. Features may comprise characteristics such as, for example, certain ranges or categories of cfDNA assay measurements, such as counts of cfDNA fragments in a biological sample obtained from a healthy and disease samples that overlap or fall within each of a set of bins (genomic windows) of a reference genome. For example, a set of features collected from a given subject at a given time point may collectively serve as a diagnostic signature, which may be indicative of an identified cancer of the subject at the given time point. Characteristics may also include labels indicating the subject's diagnostic outcome, such as for one or more cancers.

Labels may comprise outcomes such as, for example, a predicted or validated diagnosis (e.g., staging and/or tumor fraction) outcomes of the subject. Outcomes may include a characteristic associated with the cancers in the subject. For example, characteristics may be indicative of the subject having one or more cancers.

Training sets (e.g., training datasets) may be selected by random sampling of a set of data corresponding to one or more sets of subjects (e.g., retrospective and/or prospective cohorts of patients having or not having one or more cancers). Alternatively, training sets (e.g., training datasets) may be selected by proportionate sampling of a set of data corresponding to one or more sets of subjects (e.g., retrospective and/or prospective cohorts of patients having or not having one or more cancers). Training sets may be balanced across sets of data corresponding to one or more sets of subjects (e.g., patients from different clinical sites or trials). The machine learning predictor may be trained until certain predetermined conditions for accuracy or performance are satisfied, such as exhibiting particular diagnostic accuracy measures. For example, the diagnostic accuracy measure may correspond to prediction of a diagnosis, staging, or tumor fraction of one or more cancers in the subject.

Examples of diagnostic accuracy measures may include sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting the cancer.

In an aspect, the disclosure provides a method of using a classifier capable of distinguishing a population of individuals comprising:
a) assaying a plurality of classes of molecules in the biological sample, wherein the assaying provides a plurality of sets of measured values representative of the plurality of classes of molecules;
b) identifying a set of features corresponding to properties of each of the plurality of classes of molecules to be input to a machine learning or statistical model,
c) preparing a feature vector of feature values from each of the plurality of sets of measured values, each feature value corresponding to a feature of the set of features and including one or more measured values, wherein the feature vector comprises at least one feature value obtained using each set of the plurality of sets of measured values;
d) loading, into a memory of a computer system, the machine learning model comprising the classifier, the machine learning model trained using training vectors obtained from training biological samples, a first subset of the training biological samples identified as having a specified property and a second subset of the training biological samples identified as not having the specified property; and
e) inputting the feature vector into the machine learning model to obtain an output classification of whether the biological sample has the specified property, thereby distinguishing a population of individuals having the specified property.

In an aspect, the present disclosure provides a method for detecting cancer in an individual T cell receptor and/or B cell receptor expression profile in a biological sample from an individual comprising:
a) obtaining a cell-free nucleic acid from the biological sample;
b) contacting the cell-free nucleic acid with complementary oligonucleotides to regions upstream and downstream to the CDR3 domain;
wherein the complementary oligonucleotides are sequencing substantially across the CDR3 regions in the sample to generate CDR3 sequence data;
c) applying a computational analysis on the sequence data to produce the T cell receptor profile in the sample; and
d) applying the T cell receptor and/or B cell receptor profile to a machine learning model trained on T cell receptor and/or B cell receptor expression profiles to classify individuals with or without cancer.

In one embodiment, the complementary oligonucleotides are modified to permit sequencing after enzymatic conversion for methylation sequencing.

In one embodiment, the complementary oligonucleotides are selected to be complementary to regions proximal to the V-D junction and/or fully overlap the J region.

In one embodiment, the generating CDR3 nucleic acid sequence data is performed on targeted nucleic acid regions or whole genome sequencing methods

In one embodiment, the method further comprises sequencing a CDR3 domain from PBMCs from the same individual obtained at the same time as the sample of cell-free nucleic acid.

In one embodiment, the method further comprises analyzing one or more of genomic, methylomic, transcriptomic, proteomic or metabolomic information in the biological sample from the individual.

In one embodiment, the one or more of genomic, methylomic, transcriptomic, proteomic or metabolomic information in the biological sample from the individual is included in training the machine learning model trailed on T cell receptor expression.

In one embodiment, the computational analysis comprises removing non-CDR3 sequence information from the sequence data.

In one embodiment, the computational analysis comprises DNA sequence alignment, assembly, and featurization, PCA, CNN, RNN, GANN, MiXCR, TRUST, V′DJer, or DeepCAT methods.

In one embodiment, the trained machine learning model is a classifier trained to distinguish between individuals with or without cancer.

In an aspect, the present disclosure provides a method for identifying prognostic or predictive biomarkers in an individual T cell receptor and/or B cell receptor expression profile in a sample of cell-free nucleic acid from an individual comprising:
a) obtaining a sample comprising a cell-free nucleic acid;
b) contacting the cell-free nucleic acid with complementary oligonucleotides to regions upstream and downstream to the CDR3 domain;
wherein the complementary oligonucleotides are sequencing substantially across the CDR3 regions in the sample to generate CDR3 sequence data; and
c) applying a computational analysis on the sequence data to identify prognostic or predictive biomarkers in the sample.

In an aspect, the present disclosure provides a system for producing a T cell receptor and/or B cell receptor expression profile of a sample of cell-free nucleic acid from an individual comprising:
d) obtaining a sample comprising a cell-free nucleic acid;
e) contacting the cell-free nucleic acid with complementary oligonucleotides to regions upstream and downstream to the CDR3 domain wherein the complementary oligonucleotides are sequencing substantially across the CDR3 regions in the sample;
f) generating CDR3 nucleic acid sequence data;
g) applying a computational analysis on the sequence data to produce the T cell receptor and/or B cell receptor profile in the sample.

In one embodiment, the complementary oligonucleotides are modified to permit sequencing after enzymatic conversion for methylation sequencing.

In one embodiment, the modification comprises suitable modifications for enzymatic sequencing methods.

In one embodiment, the complementary oligonucleotides are selected to be complementary to regions proximal to the V-D junction and/or fully overlap the J region.

In one embodiment, the generating CDR3 nucleic acid sequence data is performed on targeted nucleic acid regions or whole genome sequencing methods

In one embodiment, the method further comprises sequencing a CDR3 domain from PBMCs from the same individual obtained at the same time as the sample of cell-free nucleic acid.

In one embodiment, the computational analysis comprises removing non-CDR3 sequence information from the sequence data.

In one embodiment, the computational analysis comprises DNA sequence alignment, assembly, and featurization, PCA, CNN, RNN, GANN, MiXCR, TRUST, V′DJer, or DeepCAT methods.

After using a trained algorithm to process the dataset, the cancer may be identified or monitored in the subject. The identification may be based at least in part on quantitative measures of sequence reads of the dataset at a panel of cancer-associated genomic loci (e.g., quantitative measures of RNA transcripts or DNA at the cancer-associated genomic loci).

Non-limiting examples of cancers that can be inferred by the disclosed methods and systems include acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloepithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney′ cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom macroglobulinemia, and Wilms Tumor, and any combination thereof.

The cancer may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the cancer by the trained algorithm may be calculated as the percentage of independent test samples (e.g., subjects having or exhibiting symptoms of cancer or subjects with negative clinical test results for the cancer) that are correctly identified or classified as having or not having the cancer.

The cancer may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the cancer using the trained algorithm may be calculated as the percentage of cell-free biological samples identified or classified as having the cancer that correspond to subjects that truly have the cancer.

The cancer may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the cancer using the trained algorithm may be calculated as the percentage of cell-free biological samples identified or classified as not having the cancer that correspond to subjects that truly do not have the cancer.

The cancer may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the cancer using the trained algorithm may be calculated as the percentage of independent test samples associated with presence of the cancer (e.g., subjects having or exhibiting symptoms of the cancer) that are correctly identified or classified as having the cancer.

The cancer may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the cancer using the trained algorithm may be calculated as the percentage of independent test samples associated with absence of the cancer (e.g., subjects with negative clinical test results for the colorectal cancer) that are correctly identified or classified as not having the cancer.

In some embodiments, the trained algorithm or classifier model may determine that the subject is at risk of colorectal cancer of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm or classifier model may determine that the subject is at risk of cancer at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

### Treatment Responsiveness

The predictive classifiers, systems, and methods described herein may be applied toward classifying populations of individuals for a number of clinical applications (e.g., based on T cell receptor and/or B cell receptor repertoire sequence profiling described herein on biological samples of individuals). Examples of such clinical applications include, detecting early-stage cancer, diagnosing cancer, classifying cancer to a particular stage of disease, determining responsiveness or resistance to a therapeutic agent for treating cancer.

The methods and systems described herein may be applied to characteristics of a cell proliferative disorder, such as grade and stage. Therefore, combinations of T cell receptor and/or B cell receptor repertoire sequences and assays may be used in the present systems and methods to predict responsiveness of cancer therapeutics across different cancer types in different tissues and classifying individuals based on treatment responsiveness. In some embodiments, the classifiers described herein are capable of stratifying a group of individuals into treatment responders and non-responders.

The present disclosure also provides a method for determining a drug target of a condition or disease of interest (e.g., genes that are relevant or important for a particular class), comprising assessing a sample obtained from an individual for the level of gene expression for at least one T cell receptor and/or B cell receptor repertoire sequences; and using a neighborhood analysis routine, determining T cell receptor and/or B cell receptor repertoire sequences that are relevant for classification of the sample, to thereby ascertain one or more drug targets relevant to the classification.

The present disclosure also provides a method for determining the efficacy of a drug designed to treat a disease class, comprising obtaining a sample from an individual having the disease class; subjecting the sample to the drug; assessing the drug-exposed sample for the level of T cell receptor and/or B cell receptor repertoire sequence expression for at least one gene; and, using a computer model built with a weighted voting scheme, classifying the drug-exposed sample into a class of the disease as a function of relative T cell receptor and/or B cell receptor repertoire sequence expression level of the sample with respect to that of the model.

The present disclosure also provides a method for determining the efficacy of a drug designed to treat a disease class, wherein an individual has been subjected to the drug, comprising obtaining a sample from the individual subjected to the drug; assessing the sample for the level of gene expression for at least one gene; and using a model built with a weighted voting scheme, classifying the sample into a class of the disease including evaluating the T cell receptor and/or B cell receptor repertoire sequence expression level of the sample as compared to T cell receptor and/or B cell receptor repertoire sequence expression level of the model.

The present disclosure also provides a method of determining whether an individual belongs to a phenotypic class (e.g., intelligence, response to a treatment, length of life, likelihood of viral infection or obesity), comprising obtaining a sample from the individual; assessing the sample for the level of gene expression for at least one gene; and using a model built with a weighted voting scheme, classifying the sample into a class of the disease including evaluating the T cell receptor and/or B cell receptor repertoire sequence expression level of the sample as compared to T cell receptor and/or B cell receptor repertoire sequence expression level of the model.

In an aspect, the systems and methods described herein that relate to classifying a population based on treatment responsiveness refer to cancers that are treated with chemotherapeutic agents of the classes DNA damaging agents, DNA repair target therapies, inhibitors of DNA damage signaling, inhibitors of DNA damage induced cell cycle arrest and inhibition of processes indirectly leading to DNA damage, but not limited to these classes. Each of these chemotherapeutic agents may be considered a “DNA-damage therapeutic agent” as the term is used herein.

Based on a patient's T cell receptor or B cell receptor repertoire sequence data, the patient may be classified into high-risk and low-risk patient groups, such as patient with a high or low risk of clinical relapse, and the results may be used to determine a course of treatment. For example, a patient determined to be a high-risk patient may be treated with adjuvant chemotherapy after surgery. For a patient deemed to be a low-risk patient, adjuvant chemotherapy may be withheld after surgery. Accordingly, the present disclosure provides, in certain aspects, a method for preparing a gene expression profile of a colon cancer tumor that is indicative of risk of recurrence.

In various examples, the classifiers described herein are capable of stratifying a population of individuals between responders and non-responders to treatment.

In another aspect, methods disclosed herein may be applied to clinical applications involving the detection or monitoring of cancer.

In some embodiments, methods disclosed herein may be applied to determine or predict response to treatment.

In some embodiments, methods disclosed herein may be applied to monitor or predict tumor load.

In some embodiments, methods disclosed herein may be applied to detect and/or predict residual tumor post-surgery.

In some embodiments, methods disclosed herein may be applied to detect and/or predict minimal residual disease post-treatment.

In some embodiments, methods disclosed herein may be applied to detect or predict relapse.

In an aspect, methods disclosed herein may be applied as a secondary screen.

In an aspect, methods disclosed herein may be applied as a primary screen.

In an aspect, methods disclosed herein may be applied to monitor cancer development.

In an aspect, methods disclosed herein may be applied to monitor or predict cancer

Upon identifying the subject as having the cancer, the subject may be optionally provided with a therapeutic intervention (e.g., prescribing an appropriate course of treatment to treat the cancer of the subject). The therapeutic intervention may comprise a prescription of an effective dose of a drug, a further testing or evaluation of the cancer, a further monitoring of the cancer, or a combination thereof. If the subject is currently being treated for the cancer with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

The quantitative measures of sequence reads of the dataset at the panel of T cell receptor or B cell receptor repertoire sequences (e.g., quantitative measures of RNA transcripts or DNA of T cell receptor or B cell receptor repertoire sequences) may be assessed over a duration of time to monitor a patient (e.g., subject who has cancer or who is being treated for cancer). In such cases, the quantitative measures of the dataset of the patient may change during the course of treatment. For example, the quantitative measures of the dataset of a patient with decreasing risk of the cancer due to an effective treatment may shift toward the profile or distribution of a healthy subject (e.g., a subject without cancer). Conversely, for example, the quantitative measures of the dataset of a patient with increasing risk of the cancer due to an ineffective treatment may shift toward the profile or distribution of a subject with higher risk of the cancer or a more advanced cancer.

The cancer of the subject may be monitored by monitoring a course of treatment for treating the cancer of the subject. The monitoring may comprise assessing the cancer of the subject at two or more time points. The assessing may be based at least on the quantitative measures of sequence reads of the dataset of T cell receptor or B cell receptor repertoire sequences (e.g., quantitative measures of RNA transcripts or DNA T cell receptor or B cell receptor repertoire sequences) comprising quantitative measures of a panel of T cell receptor or B cell receptor repertoire sequences determined at each of the two or more time points.

In some embodiments, a difference in the quantitative measures of sequence reads of the dataset of T cell receptor or B cell receptor repertoire sequences (e.g., quantitative measures of RNA transcripts or DNA of cancer-associated T cell receptor or B cell receptor repertoire sequences) comprising quantitative measures of a panel of T cell receptor or B cell receptor repertoire sequences determined between the two or more time points may be indicative of one or more clinical indications, such as (i) a diagnosis of the cancer of the subject, (ii) a prognosis of the cancer of the subject, (iii) an increased risk of the cancer of the subject, (iv) a decreased risk of the cancer of the subject, (v) an efficacy of the course of treatment for treating the cancer of the subject, and (vi) a non-efficacy of the course of treatment for treating the cancer of the subject.

In some embodiments, a difference in the quantitative measures of sequence reads of the dataset of T cell receptor or B cell receptor repertoire sequences (e.g., quantitative measures of RNA transcripts or DNA T cell receptor or B cell receptor repertoire sequences) comprising quantitative measures of a panel of T cell receptor or B cell receptor repertoire sequences determined between the two or more time points may be indicative of a diagnosis of the cancer of the subject. For example, if the cancer was not detected in the subject at an earlier time point but was detected in the subject at a later time point, then the difference is indicative of a diagnosis of the cancer of the subject. A clinical action or decision may be made based on this indication of diagnosis of the cancer of the subject, such as, for example, prescribing a new therapeutic intervention for the subject. The clinical action or decision may comprise recommending the subject for a secondary clinical test to confirm the diagnosis of the cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

In some embodiments, a difference in the quantitative measures of sequence reads of the dataset of T cell receptor or B cell receptor repertoire sequences (e.g., quantitative measures of RNA transcripts or DNA T cell receptor or B cell receptor repertoire sequences) comprising quantitative measures of a panel of cancer-associated T cell receptor or B cell receptor repertoire sequences determined between the two or more time points may be indicative of a prognosis of the cancer of the subject.

In some embodiments, a difference in the quantitative measures of sequence reads of the dataset of T cell receptor or B cell receptor repertoire sequences (e.g., quantitative measures of RNA transcripts or DNA of T cell receptor or B cell receptor repertoire sequences) comprising quantitative measures of T cell receptor or B cell receptor repertoire determined between the two or more time points may be indicative of the subject having an increased risk of the cancer. For example, if the cancer was detected in the subject both at an earlier time point and at a later time point, and if the difference is a positive difference (e.g., the quantitative measures of sequence reads of the dataset T cell receptor or B cell receptor repertoire sequences (e.g., quantitative measures of RNA transcripts or DNA at the cancer-associated genomic loci) increased from the earlier time point to the later time point), then the difference may be indicative of the subject having an increased risk of the cancer. A clinical action or decision may be made based on this indication of the increased risk of the cancer, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

In some embodiments, a difference in the quantitative measures of sequence reads of the dataset of T cell receptor or B cell receptor repertoire sequences (e.g., quantitative measures of RNA transcripts or DNA of T cell receptor or B cell receptor repertoire) comprising quantitative measures of T cell receptor or B cell receptor repertoire determined between the two or more time points may be indicative of the subject having a decreased risk of the cancer. For example, if the cancer was detected in the subject both at an earlier time point and at a later time point, and if the difference is a negative difference (e.g., the quantitative measures of sequence reads of the dataset of T cell receptor or B cell receptor repertoire sequences (e.g., quantitative measures of RNA transcripts or DNA of T cell receptor or B cell receptor repertoire) comprising quantitative measures of a panel of T cell receptor or B cell receptor repertoire sequences decreased from the earlier time point to the later time point), then the difference may be indicative of the subject having a decreased risk of the cancer. A clinical action or decision may be made based on this indication of the decreased risk of the cancer (e.g., continuing or ending a current therapeutic intervention) for the subject. The clinical action or decision may comprise recommending the subject for a secondary clinical test to confirm the decreased risk of the cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

In some embodiments, a difference in the quantitative measures of sequence reads of the dataset of T cell receptor or B cell receptor repertoire sequences (e.g., quantitative measures of RNA transcripts or DNA at the cancer-associated genomic loci) comprising quantitative measures of a panel of cancer-associated genomic loci determined between the two or more time points may be indicative of an efficacy of the course of treatment for treating the cancer of the subject. For example, if the cancer was detected in the subject at an earlier time point but was not detected in the subject at a later time point, then the difference may be indicative of an efficacy of the course of treatment for treating the cancer of the subject. A clinical action or decision may be made based on this indication of the efficacy of the course of treatment for treating the cancer of the subject, e.g., continuing or ending a current therapeutic intervention for the subject. The clinical action or decision may comprise recommending the subject for a secondary clinical test to confirm the efficacy of the course of treatment for treating the cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

In some embodiments, a difference in the quantitative measures of sequence reads of the dataset of T cell receptor or B cell receptor repertoire sequences (e.g., quantitative measures of RNA transcripts or DNA of the cancer-associated T cell receptor or B cell receptor repertoire) comprising quantitative measures of a panel of T cell receptor or B cell receptor repertoire sequences determined between the two or more time points may be indicative of a non-efficacy of the course of treatment for treating the cancer of the subject. For example, if the cancer was detected in the subject both at an earlier time point and at a later time point, and if the difference is a positive or zero difference (e.g., the quantitative measures of sequence reads of the dataset of T cell receptor or B cell receptor repertoire sequences comprising quantitative measures of T cell receptor or B cell receptor repertoire increased or remained at a constant level from the earlier time point to the later time point), and if an efficacious treatment was indicated at an earlier time point, then the difference may be indicative of a non-efficacy of the course of treatment for treating the cancer of the subject. A clinical action or decision may be made based on this indication of the non-efficacy of the course of treatment for treating the cancer of the subject, e.g., ending a current therapeutic intervention or switching to (e.g., prescribing) a different new therapeutic intervention for the subject. The clinical action or decision may comprise recommending the subject for a secondary clinical test to confirm the non-efficacy of the course of treatment for treating the cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or any combination thereof.

### VI. Kits

The present disclosure provides kits for identifying or monitoring a cancer of a subject. A kit may comprise probes for identifying a quantitative measure (e.g., indicative of a presence, absence, or relative amount) of sequences at each of a plurality of cancer-associated T cell receptor or B cell receptor sequences in a biological sample of the subject. A quantitative measure (e.g., indicative of a presence, absence, or relative amount) of sequences at each of a plurality of cancer-associated T cell receptor or B cell receptor sequences in the biological sample may be indicative of one or more cancers. The probes may be selective for the sequences at the plurality of cancer-associated T cell receptor or B cell receptor sequences in the biological sample. A kit may comprise instructions for using the probes to process the biological sample to generate datasets indicative of a quantitative measure (e.g., indicative of a presence, absence, or relative amount) of sequences at each of the plurality of cancer-associated T cell receptor or B cell receptor sequences in a biological sample of the subject.

The probes in the kit may be selective for the sequences at the plurality of cancer-associated T cell receptor or B cell receptor sequences in the biological sample. The probes in the kit may be configured to selectively enrich nucleic acid (e.g., RNA or DNA) molecules corresponding to the plurality of cancer-associated T cell receptor or B cell receptor sequences. The probes in the kit may be nucleic acid primers. The probes in the kit may have sequence complementarity with nucleic acid sequences from one or more of the plurality of cancer-associated T cell receptor or B cell receptor sequences. The plurality of cancer-associated T cell receptor or B cell receptor sequences may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct cancer-associated T cell receptor or B cell receptor sequences.

The instructions in the kit may comprise instructions to assay the biological sample using the probes that are selective for the sequences at the plurality of cancer-associated T cell receptor or B cell receptor sequences in the biological sample. These probes may be nucleic acid molecules (e.g., RNA or DNA) having sequence complementarity with nucleic acid sequences (e.g., RNA or DNA) from one or more of the plurality of cancer-associated T cell receptor or B cell receptor sequences. These nucleic acid molecules may be primers or enrichment sequences. The instructions to assay the biological sample may comprise introductions to perform array hybridization, polymerase chain reaction (PCR), or nucleic acid sequencing (e.g., DNA sequencing or RNA sequencing) to process the biological sample to generate datasets indicative of a quantitative measure (e.g., indicative of a presence, absence, or relative amount) of sequences at each of the plurality of cancer-associated T cell receptor or B cell receptor sequences in the c biological sample. A quantitative measure (e.g., indicative of a presence, absence, or relative amount) of sequences at each of a plurality of cancer-associated T cell receptor or B cell receptor sequences in the biological sample may be indicative of one or more cancers.

The instructions in the kit may comprise instructions to measure and interpret assay readouts, which may be quantified at one or more of the plurality of cancer-associated T cell receptor or B cell receptor sequences to generate the datasets indicative of a quantitative measure (e.g., indicative of a presence, absence, or relative amount) of sequences at each of the plurality of cancer-associated T cell receptor or B cell receptor sequences in the biological sample. For example, quantification of array hybridization or polymerase chain reaction (PCR) corresponding to the plurality of cancer-associated T cell receptor or B cell receptor sequences may generate the datasets indicative of a quantitative measure (e.g., indicative of a presence, absence, or relative amount) of sequences at each of the plurality of cancer-associated T cell receptor or B cell receptor sequences in the biological sample. Assay readouts may comprise quantitative PCR (qPCR) values, digital PCR (dPCR) values, digital droplet PCR (ddPCR) values, fluorescence values, etc., or normalized values thereof.

### EXAMPLES

### Example 1: Recovering Usable CDR3 Nucleic Acid Fragments and Determining Efficiency for Multiomic Analysis

A. Assessing CDR3 Probe Efficiency with Chemical Methylation-Sequencing Methods for T Cell Receptor and B Cell Receptor Sequencing Applications

The purpose of this experiment is to determine whether the hybridization probe design efficiently captured CDR3 regions in chemically converted (bisulfite-treated) DNA for methylation-specific sequencing.

Complementary probes are designed to bind upstream or downstream of CDR3 sequences in order to capture DNA fragments that contain highly variable CDR3 segments within a biological sample. For methylation-sequencing assays, probes are designed separately against both C-to-T/G-to-A converted strands of DNA and accounting for CpG's being completely methylated or unmethylated. Capture probe efficiency is ascertained by comparing unique coverage in CDR3 regions with unique coverage in control, non-variable regions of the genome (included as part of the capture panel). On-target capture rate is evaluated by determining the fraction of mapped reads originating from non-targeted regions of the genome. Biases introduced by hybridization capture can also be evaluated by comparison with whole-genome bisulfite sequencing (WGBS) performed in parallel. Variables that are optimized for hybridization capture include probe position relative to CDR3, degree of probe padding with flanking sequence, probe length, hybridization buffer, hybridization temperature/time, and wash temperatures/time. Other adjustments to probe design that increase capture of informative cfDNA fragments include mixed/degenerate bases (N's) or modified bases such as inosine.

B. Assessing CDR3 Probe Efficiency with Enzymatic Methylation-Sequencing Methods for T Cell Receptor and B Cell Receptor Sequencing Applications

The purpose of this experiment is to determine whether the hybridization probe design efficiently captured CDR3 regions in chemically converted (bisulfite-treated) DNA for methylation-specific sequencing.

Complementary probes are designed to bind upstream or downstream of CDR3 sequences in order to capture DNA fragments that contain highly variable CDR3 segments within a biological sample. For methylation-sequencing assays, probes are designed separately against both C-to-T/G-to-A converted strands of DNA and accounting for CpG's being completely methylated or unmethylated. Capture probe efficiency is ascertained by comparing unique coverage in CDR3 regions with unique coverage in control, non-variable regions of the genome (included as part of the capture panel). On-target capture rate is evaluated by determining the fraction of mapped reads originating from non-targeted regions of the genome. Biases introduced by hybridization capture can also be evaluated by comparison with whole-genome bisulfite sequencing (WGBS) performed in parallel. Variables that are optimized for hybridization capture include probe position relative to CDR3, degree of probe padding with flanking sequence, probe length, hybridization buffer, hybridization temperature/time, and wash temperatures/time. Other adjustments to probe design that increase capture of informative cfDNA fragments include mixed/degenerate bases (N's) or modified bases such as inosine.

### C. T Cell Receptor and B Cell Receptor Sequencing Methods for Immune Cell Genomic DNA, and Adaptation to Cell-Free DNA Samples

This method provides protocols for T cell receptor and B cell receptor sequencing and is also extendable to further studies against which to benchmark sequencing on cell-free DNA samples.

Immune cell genomic DNA is obtained by isolating immune cells (PBMCs, buffy coat, or enriched T cell fraction) and extracting and shearing genomic DNA. Targeted or whole-genome sequencing are performed including dsDNA library prep (with an optional hybrid capture for targeted applications) and amplification. Sequencing is performed using Illumina NovaSeq NGS platform. Sequencing is also performed using amplicon sequencing methods for comparison Critical metrics to evaluate for assay performance include clonotype diversity, on-target rate, unique coverage, and sequence duplication rate. Probe design or target capture reaction conditions can be modified to achieve target assay performance.

D. T Cell Receptor and B Cell Receptor Sequencing with Targeted Enzymatic Methylation Sequencing.

The purpose of this analysis is to perform head-to-head comparison of non-enzymatic methylation and enzymatic methylation targeted sequencing to identify and adapt any complications introduced by enzymatic methylation process such as efficiently capturing CDR3 fragments after conversion; inferring the encoded amino acid after conversion.

An established genomic sequencing method (WGBS or GEM-seq) is adapted for targeted enzymatic methylation sequencing by incorporating enzymatic methylation conversion operations and using probes designed to accommodate enzymatic methylation conversion sequencing. Critical metrics to evaluate for assay performance include clonotype diversity, on-target rate, unique coverage, and sequence duplication rate. In addition, the impact of C-to-T conversion on ability to infer the amino acids encoded by CDR3 sequences is assessed. Probe design or target capture reaction conditions can be modified to achieve target assay performance.

E. Comparison and Integration of T Cell Receptor and B Cell Receptor Sequencing on the Live Immune Cell Fraction and cfDNA Fractions of Plasma.

The purpose of this comparison is to determine the differences in CDR3 sequences represented in genomic DNA from live cells compared to CDR3 sequences represented in cell-free DNA from dead cells in the circulation. Chemical or enzymatic methylation conversion can be performed on genomic DNA isolated from buffy coat of a centrifuged blood sample. Similarly, chemical or enzymatic methylation conversion can be performed on cell-free DNA isolated from the plasma fraction of a centrifuged blood sample.

CDR3 profiles are generated from sequence information of all conditions and compared to determine the differences and additive signal between live and dead cell fractions in a blood sample.

This evaluation is repeated between normal and cancer samples to identify differences and additive signal between live and dead cell fractions in a blood samples from these individual populations. TCR and BCR repertoires can be featurized as individual clonotypes, groups of highly related clonotypes (e.g., with similar but not necessarily identical amino acids), and clonotype diversity.

### F. TCR and BCR Repertoire Profiling in Multiomic Analysis

TCR or BCR repertoires are assessed independently for classification performance (using featurizations described above) as well as in additivity models that incorporate other features, including but not limited to methylation states of cfDNA fragments, circulating protein abundances, autoantibody presence, and abundances of cell-free RNAs.

### Example 2: T Cell Receptor Sequencing (TCR-Seq)

Cancer-specific signals in cfDNA may not necessarily originate directly from tumor cells. In certain instances, a large proportion of cfDNA may be attributed to myeloid cells (leukocytes, dendritic cells, neutrophils, etc) given their abundance and relatively high rate of turnover. Thus, detecting an early immune response to cancer or neoplasia by better defining and measuring the “immune” components of cfDNA (and the cellular fraction of our samples) may be of significant interest. TCR sequencing (TCRseq), which may refer to a method to survey an individual subject's T-cell repertoire, has shown potential utility in disease (e.g., cancer) diagnosis, prognosis, and treatment response prediction.

The primary sequence determinant governing binding specificity is CDR3-β. CDR3-β spans the V-D-J junctions, and it is about 36-54 bases long (˜12-17 amino acids). This span of amino acids is typically in direct contact with the peptide presented on the MHC.

Multiplexed VDJ PCR may refer to an amplicon-based sequencing protocol that targets the VDJ (CDR3) junction. V and J (F and R) multiplexed primer sequences and concentrations are optimized to capture population diversity and account for amplification bias. Sequencing data is translated into amino acid sequences yielding 12-20mer CDR3s for downstream analysis.

Using systems and methods of the present disclosure, a sequencing and computational workflow was developed for immune repertoire analysis via analysis of clinical samples from donor subjects (e.g., obtaining and centrifuging whole blood samples, isolating buffy coat gDNA from PBMCs, preparing sequencing libraries, performing sequencing runs, and performing CDR3 and VDJ assignments). Buffy coat gDNA was stored for use as starting material in experiments aimed at comparing input gDNA amounts, commercial kits (iRepertoire), primer concentrations, primer sets, (Fr3ak-Seq), and bioinformatic tools for calling TCR sequences (MiXCR, TRUST, DeepTCR). These experiments were used for developing custom primers for assaying 1 μg of input gDNA (buffycoat), and MiXCR software for computational VDJ assignments.

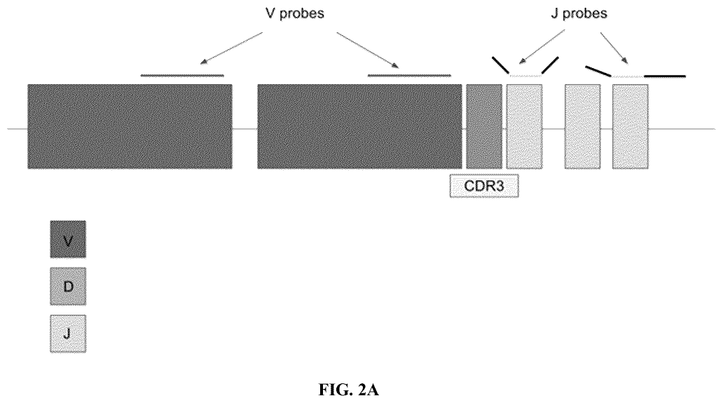

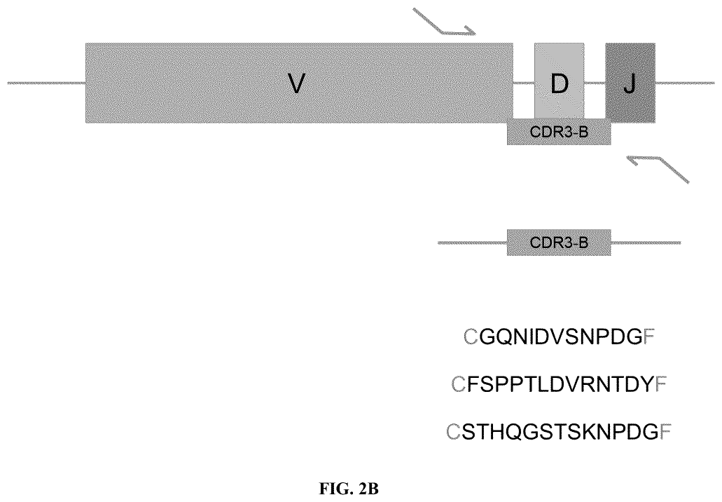

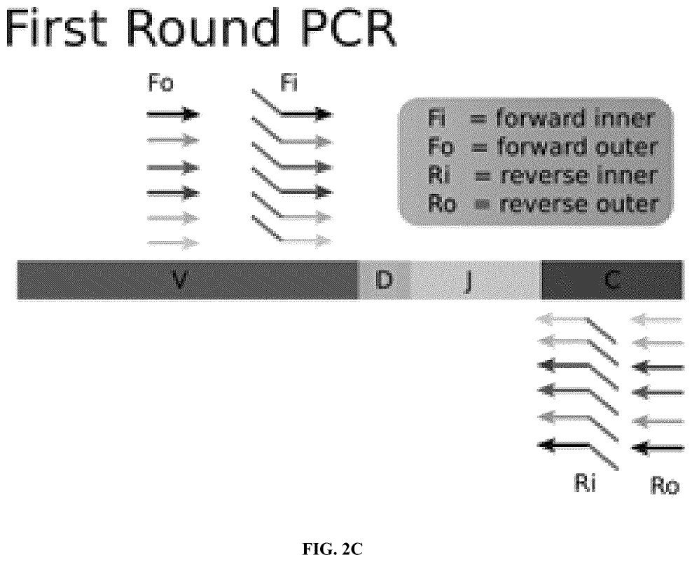

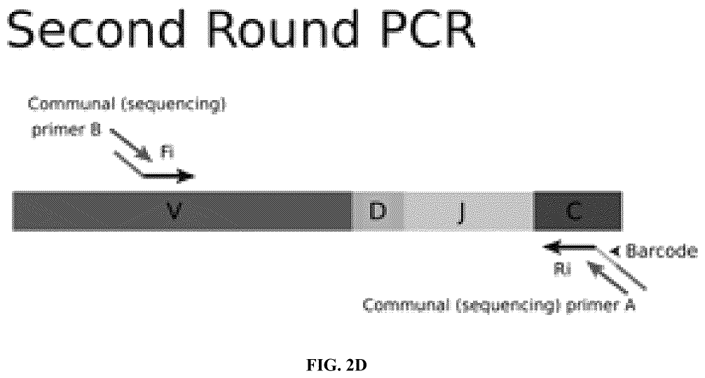

FIGS. 2A-2D provide a schematic showing VDJ region sequencing. V, D, J segments exist in germline genome, and sequencing primers across the variable CDR3 region are used to obtain substantially complete sequencing of the CDR3 region.

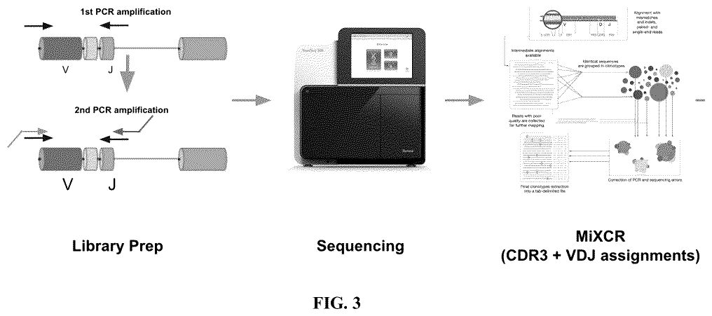

FIG. 3 provides schematics showing VDJ region sequencing, including library preparation (including a first PCR amplification and a second PCR amplification), sequencing, and performing CDR3 and VDJ assignments (e.g., using MiXCR).

Samples were assayed under PCR conditions including a first PCR amplification operation comprising amplifying the V-D-J junction of TCR-B and adding TruSeq adapters, and a second PCR amplification comprising adding p5/p7 flowcell adapters.

PCR1 (amplifying the V-D-J junction
of TCR-B and adding TruSeq adapters):

| Reagents | Volume | | Final Conc. |
|---|---|---|---|
| 2x Qiagen Multiplex PCR | 12.5 | ul | 1X |
| Master Mix | | | |
| 10 uM of Fna-TCR V primers | 2.5 | ul | 1 uM primer mix (each V |
| | | | primer ~22 nM) |
| 10 uM of Fna-TCR J primers | 2.5 | ul | 1 uM primer mix (each J |
| | | | primer ~77 nM) |
| gDNA (200 ng/ul) | 5 | ul | 1000 ng gDNA |
| Jurkat DNA | | | Variable |
| Water | Up to 25 ul | | |
| Total Volume | 25 | ul | |

First PCR cycles: 95 C for 15 mins; followed by:

25 cycles of: 95 C for 30 sec, 59C for 90 sec (according to Multiplex PCR primer kit, annealing time must be 90 sec), and 72 C for 1 min; followed by:

1 cycle of 72 C for 10 mins.

PCR2 (adding p5/p7 flowcell adapter):

| Reagents | Volume | | Final Conc. |
|---|---|---|---|
| 5x Hercules II reaction buffer | 10 | ul | 1X |
| Herculase II Fusion DNA polymerase | 0.5 | ul | n/a |
| 10 uM of Forward i5 primers (Fna-Tru-seq) | 1.25 | ul | 0.25 uM |
| 10 uM of Forward i7 primers (Fna-Tru-seq) | 1.25 | ul | 0.25 uM |
| dNTP mix (25 mM each) | 0.5 | ul | 250 nM of |
| | | | each dNTP |
| Template DNA (PCR 1 product) | 2 | ul | |
| PCR grade water | up to 50 ul | | |
| Total Volume | 50 | ul | |

Second PCR cycles: 95 C for 3 mins; followed by:

9 cycles of: 95 C for 20 sec, 65 C for 30 sec (according to Multiplex PCR primer kit, annealing time must be 90 sec), and 72 C for 1 min; followed by:

1 cycle of 72 C for 5 mins.

Computational assignment of CDR3B sequences was performed as follows. MiXCR was used for the alignment, assignment, and quantification of TCR clones in sequencing libraries. A data pipeline with a CLI interface was used to go from Illumina BCL data to MiXCR output in gcs.

Before applying the method to clinical samples, the analytical performance of the assay was evaluated using commercial gDNA, positive controls (Jurkat gDNA), and healthy donor plasma. Several experiments were carried out to this end.

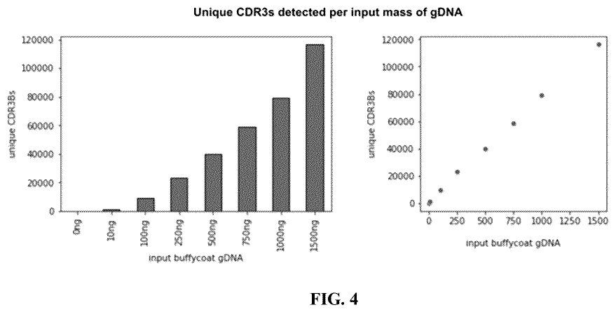

FIG. 4 provides plots showing a number of unique CDR3s detected per input mass of buffy coat genomic DNA (gDNA). Even up to 1.5 μg of input gDNA, a linear return was observed on unique clones detected, which indicates that the experiments were not approaching a sampling depth (in terms of cells) that came close to characterizing the true diversity of a subject's immune repertoire. This result may be expected given that 1 ng of buffycoat gDNA is equivalent to roughly 250 genomes. Assuming about half of the cells in buffycoat are T cells, ˜125 T cells may be expected to be present per nanogram of input. This was almost exactly observed in the data (assuming a low-level of clonality), using pooled gDNA from roughly 100 individuals. For example, at 1.5 μg input, about 185,000 cells may be expected to be sequenced.

About 120,000 unique clones were detected in the data, and roughly 80 clones per ng of input. An estimated 1E11 T cells circulate in an average individual-comprised of 1E9 unique clones (see Lythe et al., “How many TCR clonotypes does a body maintain?”, Journal of Theoretical Biology, Volume 389, 21 Jan. 2016, Pages 214-224, which is incorporated by reference herein in its entirety). A 10 μg prep run through the pipeline and sequenced exhaustively may still only capture about 800,000 unique clones, or less than 0.1% of an individual subject's repertoire.

Requisite sequencing depth was analyzed as follows. It was determined that about 50 million reads per sample was sufficient to sequence and characterize the diversity of a 1.5 μg input prep of gDNA, and that 20 million reads per sample may likely suffice for capturing the expanded or nominally functional cells (of interest) in a given sample. Rarefaction analysis using in-silico down-sampling showed that for every additional 10 million reads beyond a sequencing depth of 20 million, one may expect to detect only about 10k unique clones, most of them singlets and most of them unique to that sample.

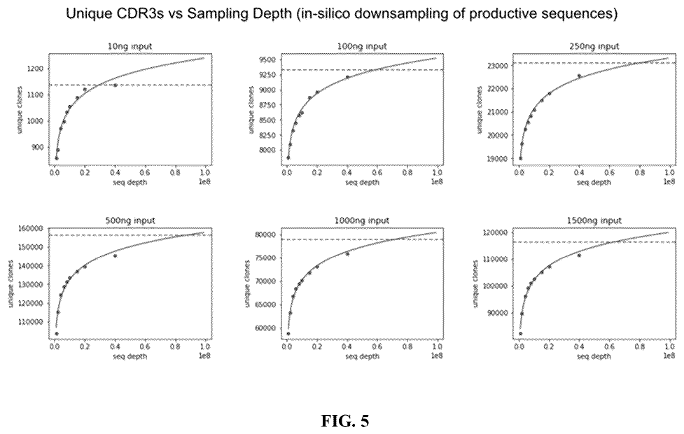

FIG. 5 provides plots showing a number of unique CDRs vs. sampling depth (e.g., in-silico downsampling of productive sequences) for various input masses (e.g., 10 ng, 100 ng, 250 ng, 500 ng, 1000 ng, and 1500 ng).

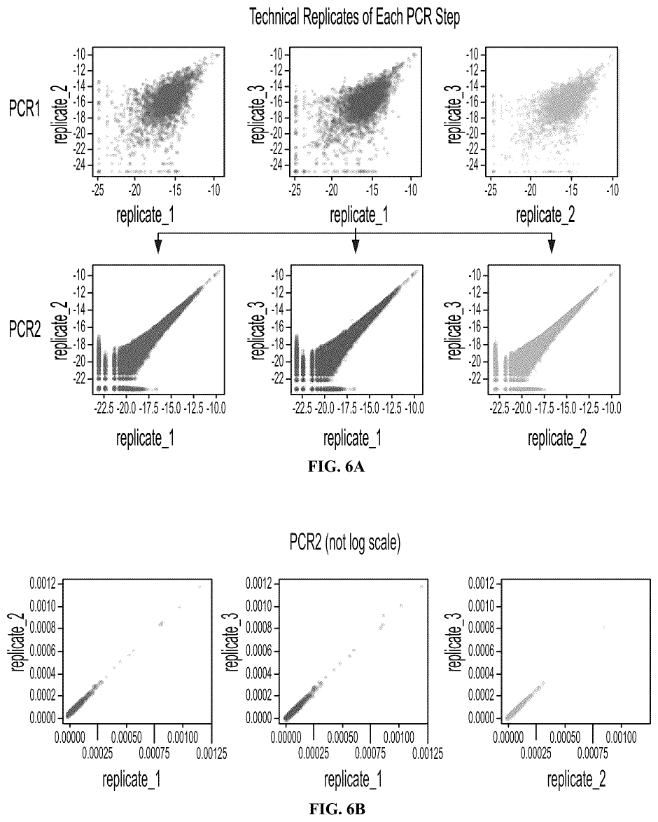

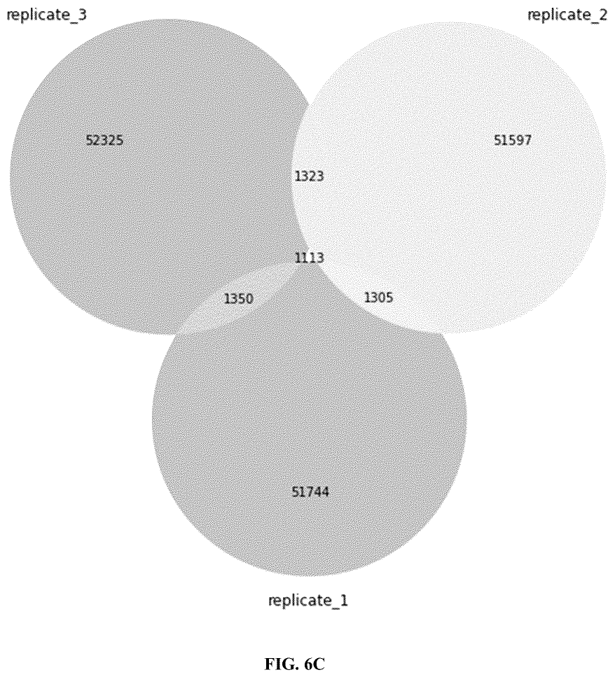

Correlation of technical replicates was performed. FIGS. 6A-6C provide plots showing comparisons between technical replicates of each of the first and second PCR amplification operations (FIGS. 6A-6B) and a Venn diagram indicating overlaps between three replicates of PCR amplification (FIG. 6C). These results demonstrated that most of the variability in the assay was due to biological signal variation.

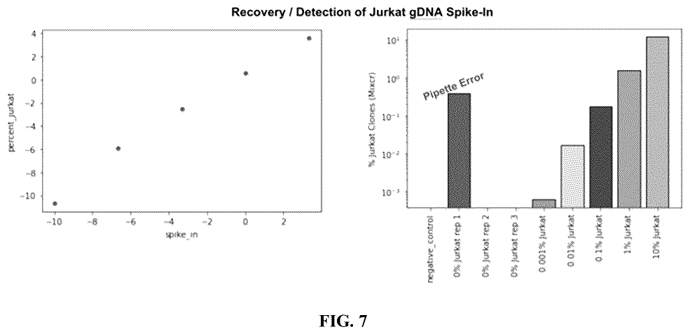

Limit of detection was assessed via spiked-in experiments. FIG. 7 provides plots showing recovery of spiked-in Jurkat gDNA (percent Junkat) and detection of spiked-in Jurkat gDNA (percent Jurkat clones detected using MiXCR). The results demonstrated the successful quantitative recovery of Jurkat clones spiked in at known fractions down to 1E-8 fraction.

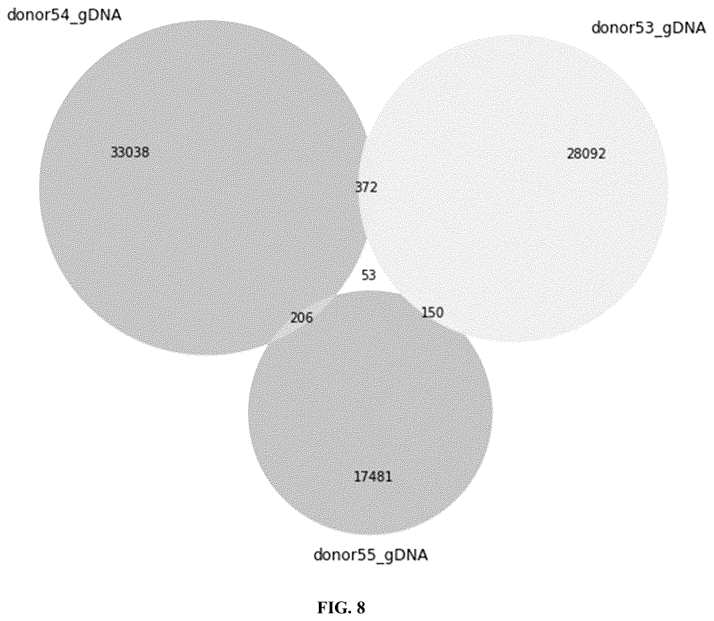

FIG. 8 provides a plot showing a Venn diagram indicating overlaps between gDNA samples from three healthy donor subjects.

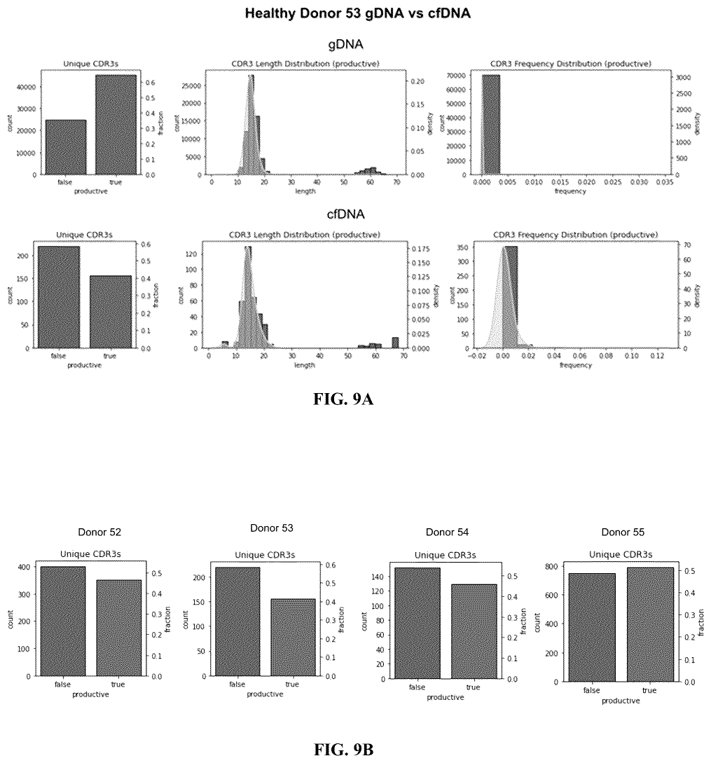

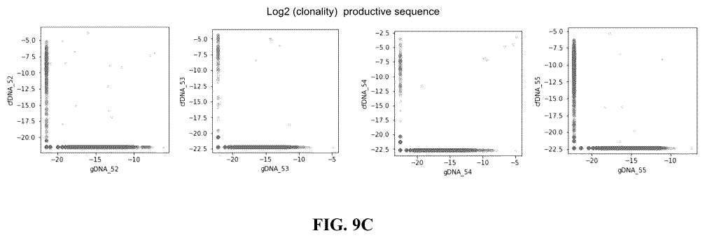

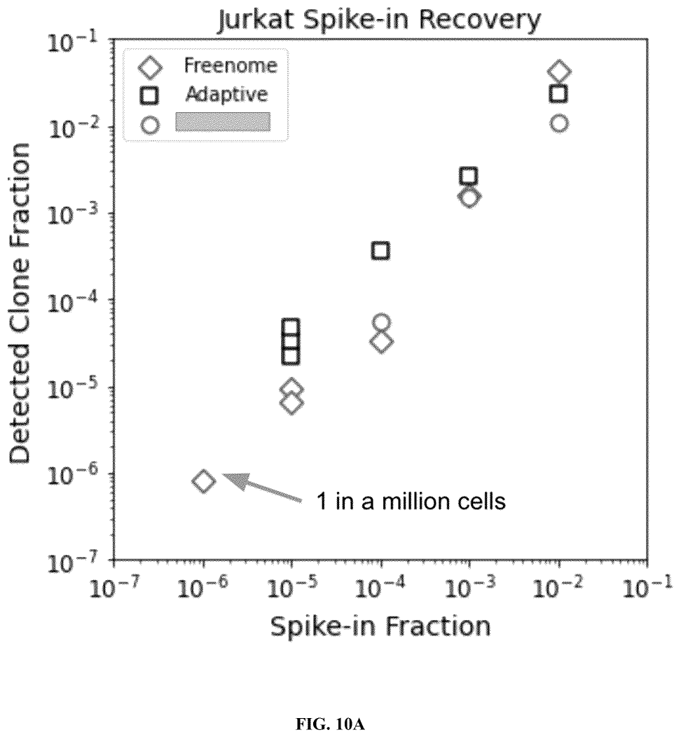

FIGS. 9A-9C provide plots showing comparisons between number of unique CDR3s, CDR3 length distribution (productive), and CDR3 frequency distribution (productive) between a healthy donor gDNA vs. cell-free DNA (FIG. 9A); comparisons between number of unique CDR3s that are productive or not productive across four donor subjects (FIG. 9B); and comparisons of productive sequences in gDNA samples across four donor subjects (FIG. 9C).

Libraries were generated libraries using the iRepertoire Kit (nested, multiplexed PCR with UMIs). These results demonstrated the low yield of cfDNA preps and increased proportion of non-productive sequences compared to paired buffy coat. About 70,000 to 100,000 T-cell clones per μg of buffy coat gDNA were observed, compared to about 400 clones per 10 ng of cfDNA; further, almost no overlap was observed between the paired samples.

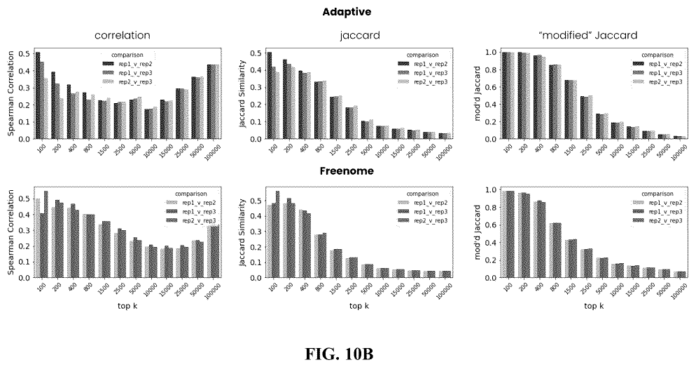

FIGS. 10A-10B provide plots showing Jurkat spike-in recovery results (detected clone fraction vs. spike-in fraction) (FIG. 10A), and comparisons between Spearman correlation, Jaccard similarity, and modified Jaccard similarity metrics between a workflow of the present disclosure (Freenome) and an alternative sequencing workflow (Adaptive) (FIG. 10B). 23 Samples from the same sources were sequenced on each platform, and comparisons were made between T-cell diversity, yield, correlation between replicates, and limit of detection of spiked in Jurkat gDNA.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-53. (canceled)

54. A method of sequencing a biological sample from a subject, the method comprising:
a) obtaining a nucleic acid from the biological sample obtained or derived from the subject;
b) contacting the nucleic acid with complementary oligonucleotides to regions upstream and downstream to a complementarity-determining region 3 (CDR3) domain, wherein the complementary oligonucleotides are sequencing substantially across the CDR3 regions in the biological sample; and
c) generating CDR3 nucleic acid sequence data from the nucleic acid.

55. The method of claim 54, wherein the biological sample comprises cell-free nucleic acid, plasma, serum, whole blood, buffy coat, single cell, or tissue.

56. The method of claim 55, wherein the biological sample comprises the cell-free nucleic acid.

57. The method of claim 55, wherein the biological sample comprises the plasma or the serum.

58. The method of claim 54, wherein the complementary oligonucleotides are modified to permit methylation sequencing after enzymatic conversion.

59. The method of claim 54, wherein the complementary oligonucleotides are designed separately against both C-to-T/G-to-A converted strands of deoxyribonucleic acid (DNA), accounting for CpG's being completely methylated or completely unmethylated.

60. The method of claim 54, wherein the complementary oligonucleotides are selected to be complementary to regions proximal to a V-D junction or to fully overlap a J region.

61. The method of claim 54, wherein generating the CDR3 nucleic acid sequence data is performed on targeted nucleic acid regions.

62. The method of claim 54, wherein generating the CDR3 nucleic acid sequence data comprises use of whole genome sequencing methods.

63. The method of claim 55, further comprising sequencing a CDR3 domain from peripheral blood mononuclear cells (PBMCs) from the subject obtained at the same time as the cell-free nucleic acid.

64. The method of claim 54, further comprising applying a computational analysis on the CDR3 nucleic acid sequence data to produce a T cell receptor (TCR) and/or a B cell receptor (BCR) profile of the subject.

65. The method of claim 64, wherein the computational analysis further comprises removing non-CDR3 sequence information from the CDR3 nucleic acid sequence data.

66. The method of claim 64, wherein the computational analysis further comprises use of DNA sequence alignment, assembly, and featurization.

67. The method of claim 64, wherein the computational analysis further comprises use of PCA, CNN, RNN, GANN, MiXCR, TRUST, V'DJer, or DeepCAT methods.

68. The method of claim 64, wherein the TCR and/or the BCR profiles are associated with a presence of a lung, a colon, a liver, an ovarian, a pancreatic, a prostate, a rectal, and/or a breast cell proliferative disorder or progression thereof.

69. The method of claim 64, further comprising detecting cancer in an individual T-cell receptor or B-cell receptor expression profile in a biological sample from a subject, wherein the detecting comprises applying a machine learning model trained on the TCR and/or BCR profiles.

70. The method of claim 69, further comprising analyzing one or more of genomic, methylomic, transcriptomic, proteomic, or metabolomic information in the biological sample from the subject.

71. The method of claim 70, wherein the one or more of genomic, methylomic, transcriptomic, proteomic, or metabolomic information in the biological sample from the subject is included in training the machine learning model trained on TCR expression.

72. The method of claim 69, wherein the trained machine learning model is a classifier trained to distinguish between subjects with or without cancer.

73. A method for identifying prognostic or predictive biomarkers in an individual T cell receptor or B cell receptor expression profile in a sample of cell-free nucleic acid from a subject, the method comprising:
a) obtaining a sample comprising a cell-free nucleic acid;
b) contacting the cell-free nucleic acid with complementary oligonucleotides to regions upstream and downstream to a complementarity-determining region 3 (CDR3) domain, wherein the complementary oligonucleotides are sequencing substantially across the CDR3 regions in the sample to generate CDR3 nucleic acid sequence data; and
c) applying a computational analysis on the CDR3 nucleic acid sequence data to identify the prognostic or predictive biomarkers in the sample.

74. A system for sequencing a sample of cell-free nucleic acid from a subject, the system comprising one or more processors and memory operatively coupled to the one or more processors, wherein the one or more processors are programmed to:
a) obtain a sample comprising a cell-free nucleic acid;
b) contact the cell-free nucleic acid with complementary oligonucleotides to regions upstream and downstream to a complementarity-determining region 3 (CDR3) domain, wherein the complementary oligonucleotides are sequencing substantially across the CDR3 regions in the sample; and
c) generate CDR3 nucleic acid sequence data from the nucleic acid.
